(19) United States
(12) Patent Application Publication
(10) Pub. No.: US 2020/0115412 A1
(43) Pub. Date: Apr. 16, 2020

(54) SEPARATION OF VANCOMYCIN AND ITS DEGRADATION PRODUCTS

(71) Applicant: SAVARA INC., Austin, TX (US)

(72) Inventors: Christopher Riley, Maryville, MO (US); Benjamin Zechinati, Hillsborough, NC (US)

(21) Appl. No.: 16/653,740

(22) Filed: Oct. 15, 2019

Related U.S. Application Data
(60) Provisional application No. 62/745,772, filed on Oct. 15, 2018.

(51) Int. Cl. C07K 1/20; C07K 9/00; B01D 15/32; B01J 20/291

(52) U.S. Cl. CPC C07K 1/20; C07K 9/008; B01J 2220/86; B01J 20/291; B01D 15/325

(57) ABSTRACT

Disclosed is a chromatographic method for separating a mixture of compounds having ionizable groups using a mobile phase comprising (a) a first mobile phase component comprising an aqueous buffer system and an organic solvent mixture miscible with water, and (b) a second mobile phase component comprising an aqueous buffer system and an organic solvent mixture miscible with water, wherein the buffer system and the solvent mixture in the first mobile phase component are different from the buffer system and the solvent mixture in the second mobile phase component and the ratio of the first mobile phase component to the second mobile phase component is varied during the separation. The method can be used for the separation of vancomycin and its degradation products.

### CROSS REFERENCE TO RELATED APPLICATIONS

This application claims the benefit of U.S. Provisional Application No. 62/745,772, filed Oct. 15, 2018, the contents of which are hereby incorporated by reference in its entirety.

### FIELD OF THE INVENTION

This invention relates to a chromatographic method for the separation of vancomycin and its degradation products.

### BACKGROUND

Vancomycin is a potent antibiotic reserved for serious drug-resistant bacterial infections including intravenous infusion for complicated infection (including MRSA) and oral administration for severe gastro-intestinal (GI) infections caused by Clostridium difficile. It was first isolated from soil bacterium (Amycolatopis orientalis) by Kornfeld in 1953, and first commercialized by Eli Lilly in 1954. Vancomycin, which is listed on the WHO's List of Essential Medicines, is a complex glycopeptide derived from N. orientalis with significant analytical challenges, including the presence of at least 12 identified impurities and a greater number of unknown impurities. Vancomycin is unstable at room temperature, hygroscopic and amorphous. Vancomycin has six ionizable groups and some of the impurities have seven. It is difficult to handle (high surface energy) and difficult to formulate. AeroVanc is a dry-powder inhalation product (DPI) under development for the treatment of methicillin resistant S. aureus (MRSA) infections in the lungs of cystic fibrosis patients. AeroVanc (vancomycin hydrochloride inhalation powder) contains the antibiotic and a single excipient, L-leucine. AeroVanc comprises a dry powder formulation which is delivered to the lungs by oral inhalation using a unit dose delivery device.

Various HPLC methods are listed in the United States Pharmacopeia (USP), European Pharmacopoeia (EP) and the British Pharmacopoeia (BP) monographs, and in the scientific literature for the assessment of vancomycin impurities. However, none of the procedures provide acceptable separations for the determination of the impurities of vancomycin in vancomycin drug substance and in AeroVanc.

US20160264620A1 and U.S. Pat. No. 9,428,291B2 teach the purification of vancomycin using reversed phase HPLC. Diana et al., HPLC, Rapid Communication Mass Spectrometry, 2006, 20(4), 685-693, and Diana et al., Journal of Chromatography A, 2003, 996(1-2), 115-131 teach the quantification of vancomycin and related substances using reversed phase HPLC with a C18 column and mass-spectrometry (MS) detection. Backes et al., Journal of Pharmaceutical and Biomedical Analysis 1998, 16 (8), 1281-1288 teaches the quantification of vancomycin and its degradation products CDP-1 Major and CDP-1 Minor using reversed phase HPLC with a C18 column. Hadwiger et al., Antimicrobial Agents and Chemotherapy, 2012, 56(6), 284-2830 and Nambiar et al., 2012, 56(26) 2819-2823, teach the quantification of vancomycin and related substances using reversed phase HPLC with a C18 column and mass-spectrometry detection.

There remains a need for improved separation of complex compounds such as vancomycin from known and unknown impurities to provide high quality medicaments.

### SUMMARY OF THE INVENTION

The invention provides method for separating a mixture of compounds each having at least one ionizable group, the method comprising: treating the mixture with reversed phase high pressure liquid chromatography on a stationary phase comprising a reversed phase chromatography column in the presence of a mobile phase comprising (a) a first mobile phase component comprising an aqueous buffer system comprising a weak acid and optionally a conjugate base, and at least one organic solvent miscible with water, and (b) a second mobile phase component comprising an aqueous buffer system comprising a conjugate base and optionally a weak acid, and a different concentration of at least one organic solvent miscible with water, wherein the ratio of weak acid to conjugate base in the first mobile phase component is different from the ratio of weak acid to conjugate base in the second mobile phase component and the ratio of the first mobile phase component to the second mobile phase component is varied during the separation.

### BRIEF DESCRIPTION OF THE FIGURES

A detailed description of various aspects, features and embodiments of the subject matter described herein is provide with reference to the accompanying drawings, which are briefly described below. The drawings are illustrative and are not necessarily drawn to scale, with some components being exaggerated for clarity. The drawings illustrate various aspects and features of the present subject matter and may illustrate one or more embodiment(s) or example(s) of the present subject matter in whole or in part. Together with the description, the drawings serve to explain the principles of the disclosed subject matter.

The patent or application file contains at least one drawing executed in color. Copies of this patent or patent application publication with color drawing(s) will be provided by the Office upon request and payment of the necessary fee.

FIG. 1A shows the structure of vancomycin and its ionization sites (pK₁, pK₂, pK₃, pK₄, pK₅, and pK₆) and degradation sites (P1, P2, P3, P4, P5 and P6).

FIG. 1B shows the effects of pH on the ionized form and net charge of vancomycin.

FIG. 2 shows a chromatogram of a separation of vancomycin using a standard method based on methods in the BP, EP and USP.

FIGS. 3A through 3D show chromatograms run at pH of 2.7, 4.5 or 7.0 with different organic solvents.

FIG. 4 summarizes the effects of changing pH and solvent on retention times for the vancomycin and major impurities, used to identify optimal compositions of the mobile phases defined by the resolution window.

FIG. 5A compares the observed (top) to predicted (bottom) retention times using an acetate/acetic acid/THF/acetonitrile mobile phase.

FIG. 5B shows the statistical correlation between observed and predicted retention time.

FIG. 6 shows a chromatogram of an assay method.

FIG. 7 shows a chromatogram of an impurity method.

FIG. 8 shows the effect of pH on the separation of vancomycin and key impurities.

FIG. 9A shows a portion of the degradation pathways of vancomycin.

FIG. 9B shows the effects of pH on the ionized form and net charge of vancomycin in the pH range of 1 to 8.

FIG. 9C shows the effects of pH on the ionized form and net charge of des-amido-isoaspartate-vancomycin in the pH range of 1 to 8.

FIG. 9D compares the effects of pH on the net charge of vancomycin and des-amido-isoaspartate-vancomycin in the pH range of 1 to 8.

FIG. 10 shows a gradient profile (top) used in an optimized separation of vancomycin and key impurities (bottom).

FIG. 11 shows how the composition of the column can affect retention time and elution order.

FIG. 12 shows a chromatogram showing separation of peaks using an isocratic region and a gradient region.

FIG. 13A-C show tables (FIG. 13A and FIG. 3B) and graph of DoE (FIG. 13C) variables for investigation.

FIG. 14A shows different gradient profiles for optimization of the gradient region.

FIG. 14B shows different solvent strengths for optimization of the isocratic region.

FIG. 15 shows chromatograms showing the effect of changing the gradient profile on a 10-cm column.

FIG. 16 shows chromatograms showing the effect of changing both the solvent strength and the gradient profile on the overall separation on a 15-cm column.

FIG. 17 shows the effect of flow rate on the separation and analysis time on a 15-cm column.

FIG. 18 shows the effect of gradient slope on the separation and analysis time on a 15-cm column.

FIG. 19 shows the effect of temperature on the separation and analysis time on a 15-cm column.

FIG. 20 shows a chromatogram of the final optimized separation on a 15-cm column.

FIG. 21 shows the separation of 0.05% CDP-1 Minor and CDP-1 Major using the method of the invention.

FIG. 22 shows the impurity profiles of degradation of vancomycin in solution (top) and solid (bottom) phases.

FIG. 23 shows the impurity profiles of degradation of vancomycin powder stored at 5° C. for 3 months.

FIG. 24 shows the impurity profiles of degradation of vancomycin powder stored at 25° C. for 3 months.

### DETAILED DESCRIPTION OF THE DISCLOSURE

The contents of all references herein are hereby incorporated by reference in their entirety.

The invention provides an analytical method designed to separate and quantify over 12 impurities and degradation products of vancomycin in vancomycin hydrochloride, an active pharmaceutical ingredient (API) and the inhalation powder form of vancomycin, AeroVanc, developed by Savara Inc.

Described herein are procedures for the determination of vancomycin impurities in vancomycin hydrochloride API and vancomycin hydrochloride inhalation powder (AeroVanc) both in bulk powder and filled into capsules.

Treating a mixture of compounds with reversed phase high pressure liquid chromatography comprises applying the mixture to one end of a column comprising a solid stationary phase and passing a liquid mobile phase through the length of the column, thereby eluting the compounds through the stationary phase to the end of the column. Because the compounds partition between the stationary phase and the mobile phase at different proportions, each of compounds in the mixture reach the end of the column at different retention times, resulting in their separation. The compounds can be detected by one or more detectors, which may also allow for quantification by comparison to standards. The retention times can be also used to identify the separated compounds by comparison to known samples of one or more of the compounds.

As used herein, the term “impurity” refers to a minor compound in a mixture with a major compound, wherein the major compound is a desirable material, such as a pharmaceutical or therapeutic agent. For example, the impurity may be present in a mixture from a finite, non-zero amount up to about 10 weight % relative to the major compound, preferably less than 5.0, 2.0, 1.0, 0.5 or 0.1 weight %. Impurities may arise from process byproducts or degradation of the major compound.

As used herein, the term “excipient” refers to an inactive or inert substance that serves as the vehicle or medium for a drug or other active substance. Pharmaceutical excipients are substances other than the active pharmaceutical ingredient (API) that have been appropriately evaluated for safety and are intentionally included in a drug delivery system or formulation. Excipients may be included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing viscosity, or enhancing solubility. Excipients can also be useful in the manufacturing process, to aid in the handling of the active substance such as by facilitating powder flowability or non-stick properties, in addition to aiding in vitro stability such as prevention of denaturation or aggregation over the expected shelf life. The selection of appropriate excipients also depends upon the active ingredient, the route of administration, the dosage form, and other factors.

As used herein, the term “gradient” refers to one or more change in the composition of the mobile phase used during a HPLC separation over time. A “step” gradient refers to a gradient wherein the mobile phase is changed to a new composition almost instantaneously and then held at the new composition for a period of time. A “linear” gradient refers to a gradient wherein the mobile phase is changed at a constant “ramp” rate or slope for a period of time. In a “non-linear” gradient, the mobile phase is changed in a non-linear fashion. For a given separation method, the overall gradient may comprise one or more of these types of gradients. Depending on the compositions of the mobile phase components used to produce the gradient, a gradient may be considered as a linear gradient for one condition or characteristic of the mobile phase and be non-linear for a second characteristic of the mobile phase. For example, a linear gradient comprising two different pH-modifying mobile phase components may result in a non-linear gradient in pH even though the solvent gradient may be linear.

The term “isocratic” refers to a mobile phase that is held constant over a period of time during a separation.

The method is suitable for the analysis of vancomycin free base, and any other salts of vancomycin, especially the hydrochloride salt. The method can be used to analyze vancomycin in various pharmaceutical formulations.

Embodiments include the following.

### Embodiment 1

The method of the Summary of the Invention wherein the first mobile phase component comprises an aqueous buffer system comprising a weak acid and no conjugate base and the second mobile phase component comprises an aqueous buffer system comprising a weak acid and a conjugate base.

### Embodiment 2

The method of the Summary of the Invention wherein the first mobile phase component comprises an aqueous buffer system comprising a weak acid and a conjugate base and the second mobile phase component comprises an aqueous buffer system comprising a conjugate base and no weak acid.

### Embodiment 3

The method of the Summary of the Invention wherein the first mobile phase component comprises an aqueous buffer system comprising a weak acid and a conjugate base and the second mobile phase component comprises an aqueous buffer system comprising a weak acid and a conjugate base.

### Embodiment 4

The method of the Summary of the Invention or any of Embodiments 1 through 3 wherein the at least one miscible organic solvent is selected from the group consisting of alcohols, acetonitrile, dioxane, tetrahydrofuran and mixtures thereof.

### Embodiment 5

The method of Embodiment 4 wherein the at least one miscible organic solvent is selected from the group consisting of tetrahydrofuran, acetonitrile and mixtures thereof.

### Embodiment 6

The method of Embodiment 5 wherein the first mobile phase component comprises tetrahydrofuran and acetonitrile and the second mobile phase component comprises tetrahydrofuran and acetonitrile in different percentages than in the first mobile phase.

### Embodiment 7

The method of the Summary of the Invention or any of Embodiments 1 through 6 wherein the buffer systems comprise formic acid, sodium formate, sodium acetate, acetic acid, ammonium formate, ammonium acetate or combinations thereof.

### Embodiment 8

The method of Embodiment 7 wherein the buffer systems comprise sodium acetate and acetic acid.

### Embodiment 9

The method of Embodiment 8 wherein the first mobile phase component comprises acetic acid and sodium acetate as buffer components.

### Embodiment 10

The method of Embodiment 9 wherein the second mobile phase component comprises acetic acid and sodium acetate as buffer components in a different ratio than in the first mobile phase.

### Embodiment 11

The method of Embodiment 9 wherein the second mobile phase component comprises sodium acetate and no acetic acid as buffer components.

### Embodiment 12

The method of the Summary of the Invention or any of Embodiments 1 through 11 wherein the pH of the first mobile phase component is controlled between 2 and 8.5.

### Embodiment 13

The method of Embodiment 12 wherein the pH of the first mobile phase component is controlled between 2.5 and 7.

### Embodiment 14

The method of Embodiment 12 wherein the pH of the first mobile phase component is controlled between 3 and 5.

### Embodiment 15

The method of Embodiment 12 wherein the pH of the first mobile phase component is controlled between 3.5 and 4.5.

### Embodiment 16

The method of the Summary of the Invention or any of Embodiments 1 through 15 wherein the pH of the second mobile phase component is controlled between 2.5 and 9.5.

### Embodiment 17

The method of Embodiment 16 wherein the pH of the second mobile phase component is controlled between 6.0 and 9.0.

### Embodiment 18

The method of Embodiment 16 wherein the pH of the second mobile phase component is controlled between 7.5 and 9.0.

### Embodiment 19

The method of the Summary of the Invention or any of Embodiments 1 through 18 wherein the reversed phase chromatography column comprises a stationary phase comprising silica gel particles having a chemically modified surface comprising octadecyl-pentafluorophenyl-silyl (C18 PFP) moieties.

### Embodiment 20

The method of the Summary of the Invention or any of Embodiments 1 through 19 wherein the second mobile phase component comprises 0% of the total mobile phase for an initial period of time and is then increased in one or more linear ramps to comprise a major portion (at least 50%; at least 60% at least 70% or at least 80%) of the total mobile phase over a second period of time.

### Embodiment 21

The method of Embodiment 20 wherein the second mobile phase is 0% of the total mobile phase for a third period of time.

### Embodiment 22

The method of the Summary of the Invention or any of Embodiments 1 through 21 wherein the second mobile phase component comprises 0% of the total mobile phase for an initial period of time and is then increased in a single step to comprise a major portion (at least 50%; at least 60% at least 70% or at least 80%) of the total mobile phase over a second period of time.

### Embodiment 23

The method of Embodiment 22 wherein the second mobile phase is 0% of the total mobile phase for a third period of time.

### Embodiment 24

The method of the Summary of the Invention or any of Embodiments 1 through 23 comprising quantifying the amount of each of the compounds in the mixture.

### Embodiment 25

The method of the Summary of the Invention or any of Embodiments 1 through 24 comprising identifying at least one impurity in the mixture.

### Embodiment 26

The method of the Summary of the Invention or any of Embodiments 1 through 25 comprising detecting the compounds of the mixture using an ultraviolet-visible light detector.

### Embodiment 27

The method of the Summary of the Invention or any of Embodiments 1 through 25 comprising detecting the compounds of the mixture using a diode-array detector.

### Embodiment 28

The method of the Summary of the Invention or any of Embodiments 1 through 25 comprising detecting the compounds of the mixture using a mass spectrometer.

### Embodiment 29

The method of the Summary of the Invention or any of Embodiments 1 through 28 wherein at least one of the compounds in the mixture has at least two ionizable groups.

### Embodiment 30

The method of Embodiment 29 wherein each of the compounds in the mixture has at least two ionizable groups.

### Embodiment 31

The method of the Summary of the Invention or any of Embodiments 1 through 28 wherein at least one of the compounds in the mixture has at least three ionizable groups.

### Embodiment 32

The method of Embodiment 31 wherein each of the compounds in the mixture has at least three ionizable groups.

### Embodiment 33

The method of the Summary of the Invention or any of Embodiments 1 through 28 wherein at least one of the compounds in the mixture has at least four ionizable groups.

### Embodiment 34

The method of Embodiment 33 wherein each of the compounds in the mixture has at least four ionizable groups.

### Embodiment 35

The method of the Summary of the Invention or any of Embodiments 1 through 28 wherein at least one of the compounds in the mixture has at least five ionizable groups.

### Embodiment 36

The method of the Summary of the Invention or any of Embodiments 1 through 28 wherein at least one of the compounds in the mixture has at least six ionizable groups.

### Embodiment 37

The method of the Summary of the Invention or any of Embodiments 1 through 36 wherein the mixture of compounds comprises a glycopeptide antibiotic and one or more impurities or degradation products.

### Embodiment 38

The method of Embodiment 37 wherein the mixture of compounds comprises vancomycin or vancomycin hydrochloride, and one or more impurities.

### Embodiment 39

The method of Embodiment 38 wherein the mixture further comprises leucine.

### Embodiment 40

The method of Embodiment 39 wherein at least one impurity in a vancomycin composition can be measured in the range of 0.05% to 10.0% relative to vancomycin (i.e. at least one impurity in a vancomycin mixture is present in the range of 0.05% to 10.0% relative to vancomycin).

Embodiments of this invention, including Embodiments 1 through 40 above as well as any other embodiments described herein, can be combined in any manner, and the descriptions of variables in Embodiments 1-40 above as well as any other embodiments described herein, and any combination thereof, pertain to the methods of the present invention. Notably, any of the embodiments above can be further embodied by any other embodiment above, such as wherein any embodiment above can further embody any of the embodiments preceding it.

A notable method for separating a mixture of compounds each having at least two ionizable groups comprises treating the mixture with reversed phase high pressure liquid chromatography on a stationary phase comprising a reversed phase chromatography column in the presence of a variable mobile phase comprising (a) a first mobile phase component comprising an aqueous sodium acetate/acetic acid buffer system and at least one organic solvent miscible with water, and (b) a second mobile phase comprising an aqueous sodium acetate/acetic acid buffer system, and a higher concentration of at least one organic solvent miscible with water, wherein the ratio of sodium acetate/acetic acid in the first mobile phase component is different from the sodium acetate/acetic acid in the second mobile phase component and the ratio of the first mobile phase component to the second mobile phase component is varied during the separation.

Vancomycin and vancomycin salts, alone or in combination with other antibiotics, are useful in treating staphylococcal, streptococcal and enterococcal infections or diphtherial endocarditis. An indication of oral vancomycin therapy includes the treatment of pseudomembranous colitis caused by staphylococci when it is unresponsive to vancomycin for injection. Vancomycin for injection may be applicable to all of the other indications. An inhalable powder form of vancomycin currently in development, AeroVanc, may be useful for treatment of methicillin resistant S. aureus (MRSA) infections in the lungs of cystic fibrosis patients.

The vancomycin molecule is composed of two basic structures, including a disaccharide group, α-o-vancosamine-β-o-glucosyl, and a heptapeptide backbone. The structure of vancomycin determines its instability and the ease with which it may be degraded under acidic conditions, alkaline conditions, and/or high temperature conditions. Vancomycin is produced by microbial fermentation, which may produce impurities with structures different from vancomycin. The impurities include process impurities and degradation products. Sometimes, impurities and degradant products may have different pharmacologic properties and toxicologic properties from the main ingredient. Thus, it is important to be able to characterize the purity of various vancomycin preparations. Table 1 summarizes vancomycin and known impurities contained in commercial preparations. It is important to be able to separate these peaks to monitor purity and/or degradation of vancomycin in pharmaceutical products to ensure good product quality, safety and efficacy.

TABLE 1
Structures of Vancomycin and Known Impurities (Process Impurities and Degradation Products).

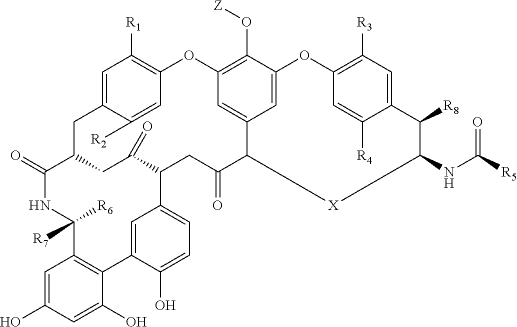

| | Code | Z | X | R₁ | R₂ | R₃ | R₄ | R₅ | R₆ | R₇ | R₈ |
|---|---|---|---|---|---|---|---|---|---|---|---|
| Vancomycin | V | 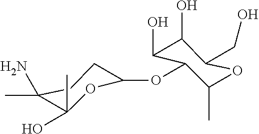 | 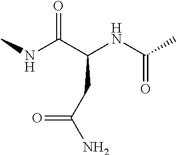 | —Cl | —H | —Cl | —H | 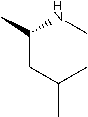 | —H | —COOH | —OH |

PROCESS IMPURITIES

| | | | | | | | | | | | |
|---|---|---|---|---|---|---|---|---|---|---|---|
| De-((L-asn)-L-gln-vancomycin (EP Impurity B) | S1 | 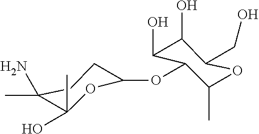 | 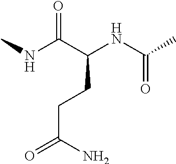 | —Cl | —H | —Cl | —H | 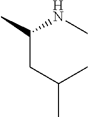 | —H | —COOH | —OH |
| N-demethyl-vancomycin (EP Impurity A) | S2 | 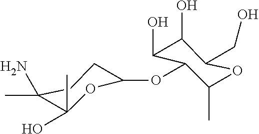 | 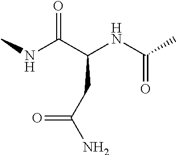 | —Cl | —H | —Cl | —H | 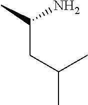 | —H | —COOH | —OH |
| Monodechloro-(2)-vancomycin | S3 | 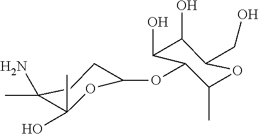 | 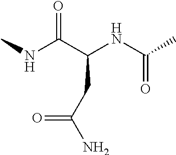 | —Cl | —H | —Cl | —H | 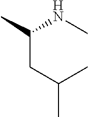 | —H | —COOH | —OH |
| Cis-chloro-(2)-vancomycin | S4 | 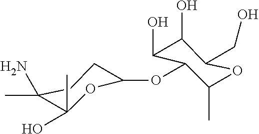 | 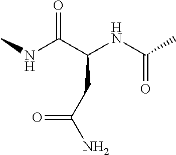 | —Cl | —H | —H | —C1 | 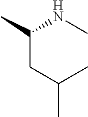 | —H | —COOH | —OH |
| Monodechloro-(6)-vancomycin | S5 | 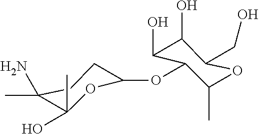 | 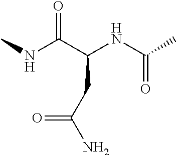 | —H | —H | —Cl | —H | 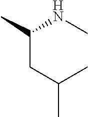 | —H | —COOH | —OH |
| N-methyl-vancomycin | S6 | 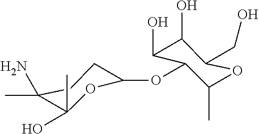 | 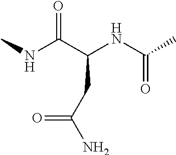 | —Cl | —H | —Cl | —H | 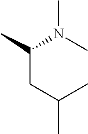 | —H | —COOH | —OH |
| De-oxy-(2)-vancomycin | — | 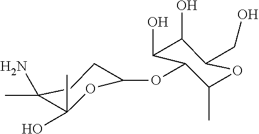 | 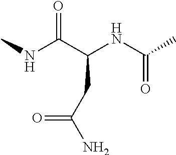 | —Cl | —H | —Cl | —H | 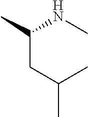 | —H | —COOH | —H |
| De-(N-methyl-D-Leu)-D- phenylalanine-vancomycin | — | 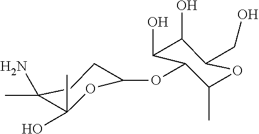 | 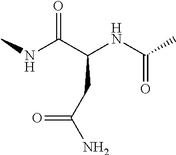 | —Cl | —H | —Cl | —H | 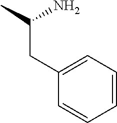 | —H | —COOH | —OH |

DEGRADATION PRODUCTS

| | | | | | | | | | | | |
|---|---|---|---|---|---|---|---|---|---|---|---|
| Des-(amido)-succinimido- vancomycin | P1 | 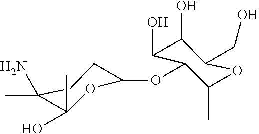 | 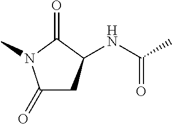 | —Cl | —H | —Cl | —H | 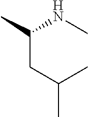 | —H | —COOH | —OH |
| Des-(amido)-aspartyl-vancomycin (CDP-1 minor) | P2 | 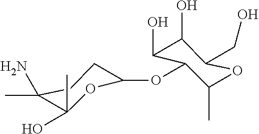 | 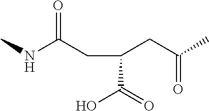 | —Cl | —H | —Cl | —H | 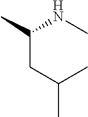 | —H | —COOH | —OH |
| Des-(amido)-aspartyl-vancomycin (CDP-1 Major) | P3 | 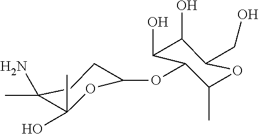 | 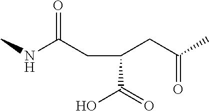 | —Cl | —H | —H | —Cl | 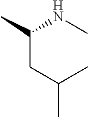 | —H | —COOH | —OH |
| Desvancosamino-vancomycin (EP Impurity D) | P4 | 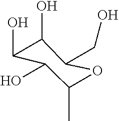 | 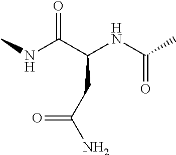 | —Cl | —H | —Cl | —H | 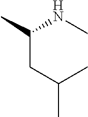 | —H | —COOH | —OH |
| Epi-(26R)-vancomycin | P5 | 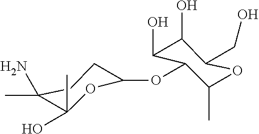 | 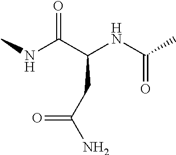 | —Cl | —H | —Cl | —H | 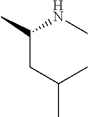 | —COOH | —H | —OH |
| Agluco-vancomycin (EP Impurity C) | P6 | —OH | 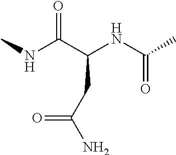 | —Cl | —H | —Cl | —H | 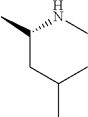 | —H | —COOH | —OH |

Desvancosaminyl-vancomycin and agluco-vancomycin are degradation products that result from the loss of the disaccharide moiety and the vancosamine sugar, respectively, by hydrolysis. Vancomycin can degrade to des-amido-succinimido-vancomycin by deamidation (Harris et al., Journal of the American Chemical Society, 1983 105 (23), 6915-6922 and Antipas et al., International Journal of Pharmaceutics, 1994, 109, 261-269). Des-amido-succinimido-vancomycin can further degrade to a mixture of two isomers of des-amido-isoaspartyl-vancomycin (CDP-1 Minor and CDP-1 Major). A number of other degradation or process impurities can be found in preparations of vancomycin hydrochloride, some of which are shown in Table 1.

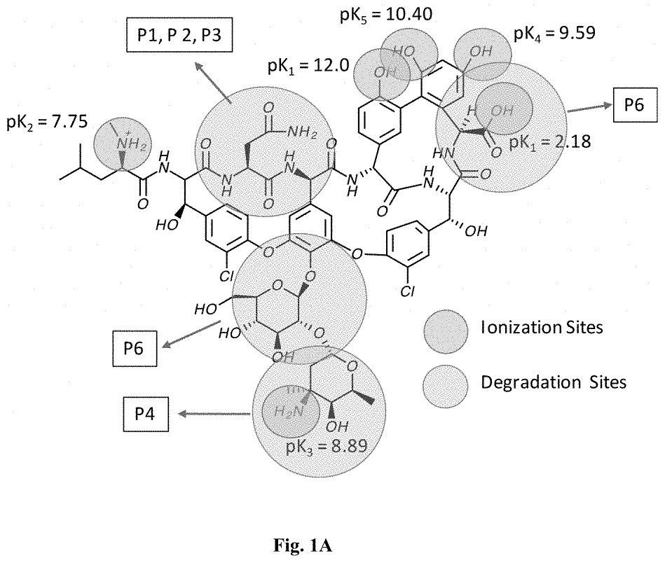

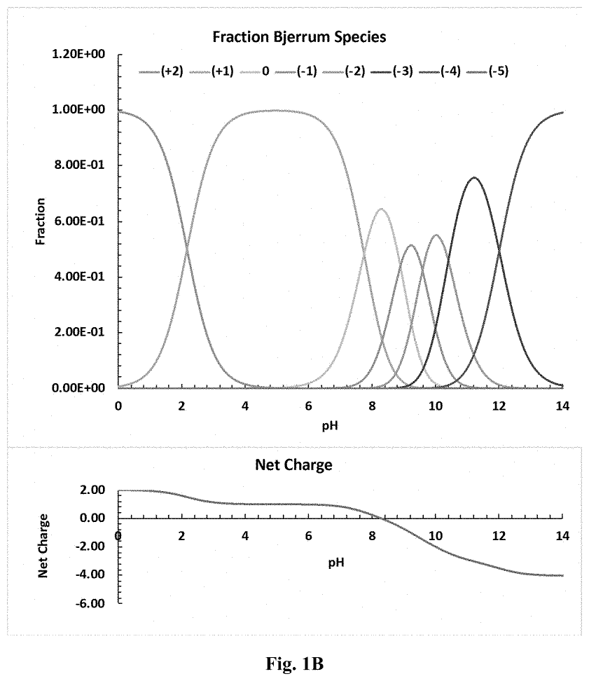

FIG. 1A shows the chemical structure of vancomycin and its ionization sites and six key degradation sites. P1, P2 and P3 indicate sites of deamidation, followed by hydrolysis. P4 and P6 show sites of hydrolysis and P5 shows sites of epimerization. The six ionization pKa values are indicated in FIG. 1A. FIG. 1B shows the relationship between pH and ionizable forms and net charge on vancomycin. Six ionization states are observed for vancomcyin, giving rise to seven possible net charges on the molecule ranging from +2 (at low pH) to −4 (at high pH). There is a plateau region between 3.5 and 5.5 where the net charge on vancomycin is constant at +1. However, the net charges on some impurities may change dramatically in this pH region due to the presence of other functional groups, such as an isoasparate group, which has a pKa value of approximately 4.5. Therefore, exploring pH adjustment in this region can prove useful, since the pH used may provide selective changes in retention time of some peaks without affecting others.

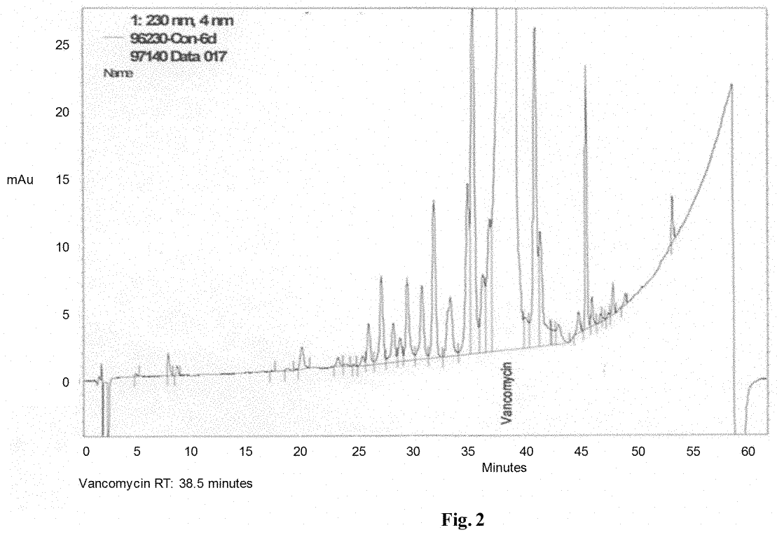

FIG. 2 shows a chromatogram of a prior separation of AeroVanc vancomycin and various impurities using a standard method based on USP, EP, BP and literature procedures. This HPLC method involved a Phenomenex Luna PFP(2) column (15 cm long, 4.6 mm in diameter packed with 3 μm-diameter particles), eluted with mobile phase comprised of trifluoroacetic acid/methanol. This method has several problems, including no identification of peaks, run-to-run variability of retention time of peaks, switching of retention time order of certain pairs of peaks, incomplete separation of many overlapping peaks and several peaks representing impurities are seen to overlap with the vancomycin peak. These factors made analysis of purity and the extent of degradation very difficult.

This disclosure discusses the importance of design-of-experiments (DOE) combined with a good understanding of the physicochemical properties of vancomycin (especially the relationship between the charge of the molecule and the mobile phase pH) in the rational development of a robust analytical method for the separation of complex mixtures in a solid dosage form. A reversed HPLC column packed with an octadecyl-penta-fluorophenyl-silyl-bonded phase (C18-PFP) such as available from ACE (Advanced Chromatography Technologies, Ltd., Aberdeen, Scotland) proved to be particularly suitable due to its high functional group selectivity and its ability to separate positional and geometric isomers. The first design-of-experiments (DOE-1) involving the investigation of pH (2.7 to 7.0), and organic solvent composition (acetonitrile, THF and methanol) and concentration (0-100%) identified the optimum conditions for separation of vancomycin and impurities on a 10-cm long C18-PFP column.

### Mobile-Phase Composition

Careful control of mobile-phase pH and solvent composition is important to the HPLC separation in the analysis of the impurities.

Factors to consider when developing a robust analytical method for separating complex mixtures include the following.

Because the compounds in the mixture may have multiple ionizable groups, the mobile phase pH needs to be controlled to provide a pH region that provides the best separation depending on the pKa values of the various ionizable groups.

Organic modifier solvent type and concentration controls selectivity (elution order) and overall retention time. There is a limited number of solvents to choose from because they should be miscible with water. Methanol, tetrahydrofuran (THF) and acetonitrile are preferred solvents in reversed phase HPLC.

Selecting the ideal combination of mobile phase pH and modifier is challenging. Commercial software packages are available but they are expensive and do not consider small differences in chemical structures of the drug and impurities. It is important to have knowledge of chromatographic theory, chromatographic practice and physical organic chemistry to develop methods for each separation problem, especially separations of very complex mixtures of structurally very similar compounds, some of which the structures of some compounds are known and structures of other compounds are unknown. Good separation of closely related impurities is necessary for sensitive, accurate and precise measurements.

The composition of the HPLC column also needs to be considered in combination with the mobile phase composition. For developing the methods described herein, we chose a PFP-C18 column because of the ability of the stationary phase to separate closely related compounds, especially isomers.

The mobile phases used in this invention comprise a combination of aqueous buffers and water-miscible organic solvents selected from alcohols, such as methanol (MeOH), ethanol, propanol, isopropanol, acetonitrile (MeCN), dioxane and tetrahydrofuran (THF).

Buffer solutions may include formic acid, providing a pH of about 2.7, sodium acetate/acetic acid mixture, providing a pH of about 4.5, and ammonium formate, providing a pH of about 7.0. Another aqueous system may comprise ammonium acetate. Mobile phases comprise mixtures of the buffer solutions and one or more of the organic solvents.

The mobile phases can be selected to provide baseline resolution of various impurities by modifying the amounts of buffer and organic solvents in each mobile phase. During the chromatographic separation, the mobile phase(s) may be run under isocratic conditions (constant mobile phase composition), gradient conditions (variable mobile phase composition) or a combination of both.

The precise control of pH is important for separating compounds with one or more ionizable groups (notably two or more), such as vancomycin, which could significantly alter their interaction with the column depending on their charged state. A pH buffer is either an aqueous mixture of a weak acid and its conjugate base or a mixture of a weak base and its conjugate acid. In this invention, two or more mobile phase compositions having different organic-solvent compositions and different pH values are used. This allows the solvent composition and the pH to be decoupled in the combined mobile phase. The different mobile phase compositions are mixed together by programming the HPLC pumps in order to change both the organic-solvent strength and the pH of the combined mobile phase flowing through the column. As a result, the pH of the combined mobile phase and the solvent strength depend on the ratio of the two (or more) mobile phase compositions at any point during the chromatographic run. In this invention the ratio of the weak acid and the conjugate base is changed during the gradient region of the separation. The pH of the combined mobile phase flowing through the column is determined by the pH of the first mobile phase composition and the pH of the second mobile phase composition. The rate of change of pH of the combined mobile phase is controllable because the pH is related to the pKa value of the buffer and the ratio of the concentrations of the two components in the buffer. Similarly, the solvent strength of the combined mobile phase also changes based on the ratio of the first mobile phase composition and the second mobile phase composition. However, solvent strength is an additive property, while pH is logarithmic. Even though the rate of change of the organic solvents by changing the ratio of the first and second mobile phase compositions may be linear, the rate of change of pH with time is non-linear due to the complex relationship between pH and the ratio of the two concentrations of the components in the buffer.

By proper selection of the pH and organic solvent components of the first and second mobile phase compositions, and their ratio during a chromatography run, the pH and solvent strength can be customized to provide effective separations of complex mixtures. For example, two mobile phases compositions used in this invention are acetate buffer systems wherein the pH of the first mobile phase component is kept around 3 to 5, preferably from about 3.9 to 4.5, or about 4.0 to about 4.3; and the pH of the second mobile phase component is kept around 3 to 10, preferably from about 6.0 to 9.5, or about 8.0 to 9.0, allowing one to modify the pH of the combined mobile phase by changing the ratio of the two mobile phases. The organic solvent strength (composition) is also different in the first and second compositions, so that the solvent strength of the combined mobile phase can be modified, but at a different rate than the pH. This invention provides a way to change multiple components of a mobile phase composition using only two mobile phase compositions.

Examples of using this technique are demonstrated by the following separations of vancomycin from associated impurities. In order to develop an improved separation method for vancomycin, we studied separations by varying factors including pH and solvent under gradient conditions, using a statistical design of experiments and two mobile phases containing an aqueous buffer (pH 2.7, 4.5 or 7.0) and one of three solvents: acetonitrile, methanol or tetrahydrofuran (THF). The column temperature, gradient shape and flow rate were held constant. An improved method was developed by a design of experiments (DOE-1) investigation in which we conducted six experiments in a two-thirds factorial design (six binary combinations of one solvent and one buffer). General chromatographic parameters for the first improved method for the vancomycin impurities studied in DOE-1 are summarized below.

TABLE 2
Design of Experiment (DOE-1) Conditions
Parameters

| | | | |
|---|---|---|---|
| Temperature: | 30° C. | Run Time: | 60 min |
| Column | | Gradient Profile | |
| Stationary Phase: | ACE C-18-PFP | Start: | 0% B |
| Particle size: | 2 μm | End: | 100% B |
| Length: | 10 cm | Ramp: | 1.67%/min |
| Width: | 2.1 mm | | |
| Injection volume: | 30 μL | | |
| Detection: | 230 nm | | |
| Mobile Phase | | Flow Rate: | 1 mL/min |

| | |
|---|---|
| Solvent A: | Buffer/MeOH, THF or MeCN) (80:20) |
| Solvent B: | Buffer/MeOH, THF or MeCN (20:80) |

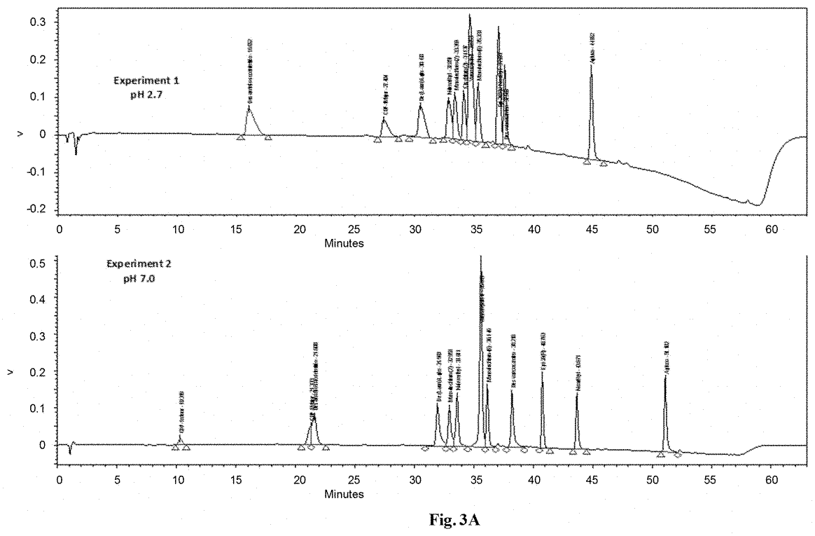

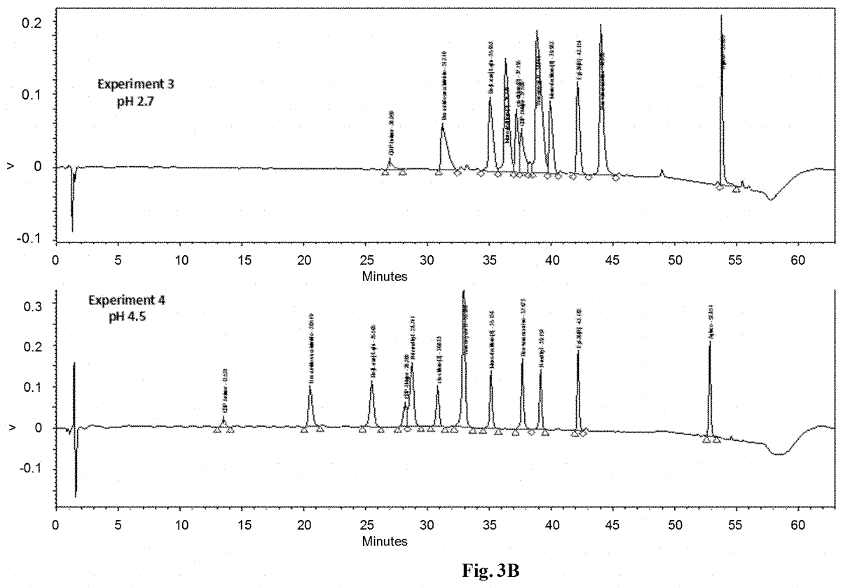

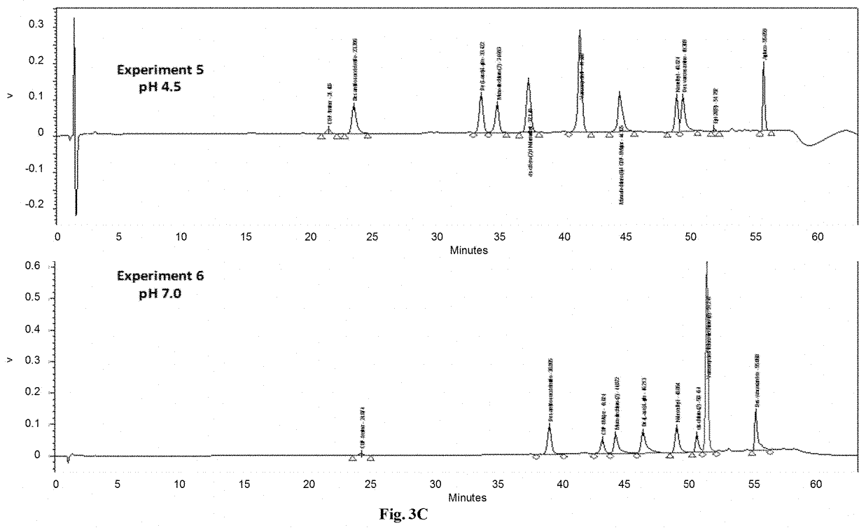

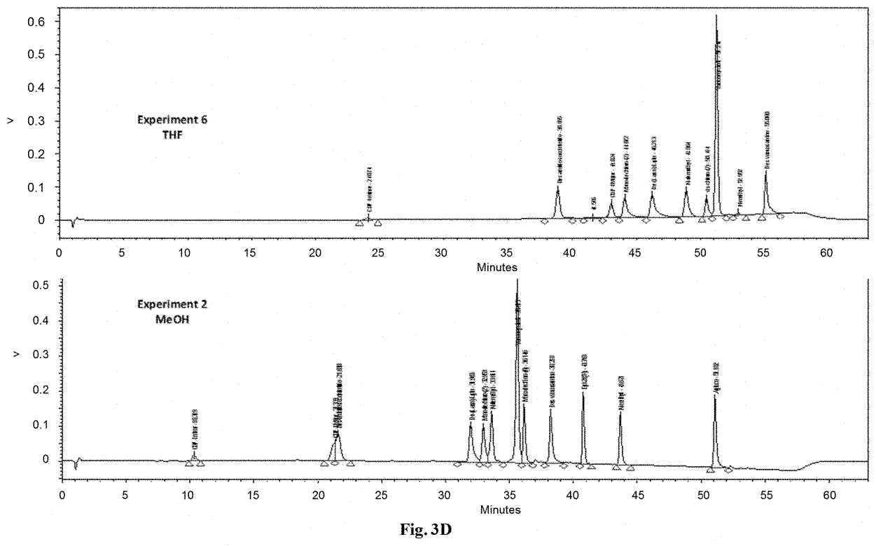

FIGS. 3A through 3D show chromatograms run at pH of 2.7, 4.5 and 7.0 with different organic solvents. FIG. 3A compares separation of vancomycin and 12 major impurities in two of the six design of experiments run, showing the effect of changing pH in a binary gradient containing buffer and methanol (experiments 1 and 2, respectively). FIG. 3B compares experiments showing the effect of changing pH in a binary gradient containing buffer and acetonitrile (experiments 3 and 4, respectively). FIG. 3C compares experiments showing the effect of changing pH in a binary gradient containing buffer and tetrahydrofuran (THF) (experiments 5 and 6, respectively). FIG. 3D compares experiments showing the effect of changing solvent in a binary gradient containing pH 7.0 buffer and either methanol and tetrahydrofuran (THF) (experiments 6 and 2, respectively). With a few exceptions the order of elution is generally: P2 (CDP-1minor), P1 (imide), P3 (CDP-1Major), P4 (desvanco), P5 (epi), P6 (agluco), as shown in Table 1. Notably, CDP-1minor, CDP-1Major and desvanco always elute in the same order with respect to each other but can elute in a different order with respect to the other peaks depending on the pH of the mobile phase. The designation P refers to the degradation products of vancomycin and the designation S indicates other known impurities that are not degradants.

Choice of solvent and pH also affected peak shape, retention times and retention-time order. Chromatography at pH of 2.7 exhibited peak tailing compared to separations at higher pH (FIG. 3A and FIG. 3B). In some instances, the order of elution changed with different pH and solvent type. FIG. 3B shows dramatic shifts in retention-time order with change of pH in acetonitrile. Similar effects were observed in methanol (FIG. 3A) and THF (FIG. 3C). The combination of low pH and methanol showed significant peak tailing, eliminating that combination from further consideration (FIG. 3A). Low pH in acetonitrile gave poor peak shape, also eliminating that combination from further consideration (FIG. 3B), but very good peak shape was observed with pH 4.5 in acetonitrile (FIG. 3B). Methanol, THF and acetonitrile all gave good peak shape at pH 4.5 and pH 7.0 (experiment 2, experiment 4, experiment 5 and experiment 6).

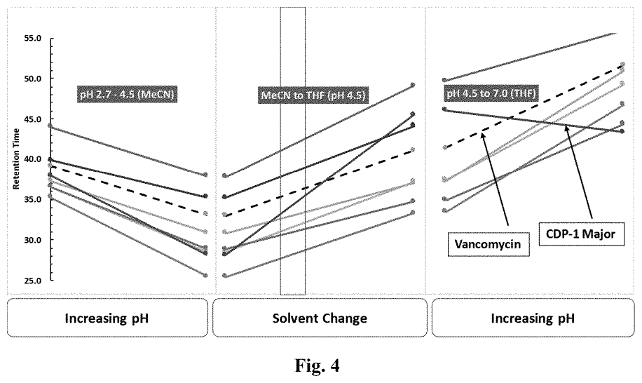

FIG. 4 summarizes the effects of changing pH and solvent on retention times for the vancomycin and major impurities. No combination of a buffer (pH 2.7, 4.5 or 7) plus one organic solvent (a binary solvent system) produced baseline separation of all known impurities and vancomycin. FIG. 4 shows retention times of vancomycin and key difficult-to-resolve peaks under roughly isoelutropic conditions (approximately constant solvent strength). Note how the retention time of CDP-1 Major changes dramatically relative to the others due to this compound having an additional pKa of approximately 4.5 (the pH of mobile phase), arising from the replacement of the neutral amino acid, glutamine, with the acidic amino acid, iso-aspartic acid. The rectangle predicts a possible resolution window with a ternary system of acetate buffer (pH of around 4.5)/THF/acetonitrile. The retention times of vancomycin and impurities in mobile phases containing an aqueous buffer and one, two or three solvents were predicted (t_{predicted}) from the formula:

t_{predicted}=f_{MeOH}t_{MeOH}+f_{MeCN}t_{MeCN}+f_{THF}t_{THF}

where t_{MeOH}, t_{MeCN}, and t_{THF} are the retention times in the mobile phase containing buffer and one solvent and f_{MeOH}, f_{MeCN} and f_{MeOH}, are the volume fractions of the solvents in mobile phase containing one, two or three solvents.

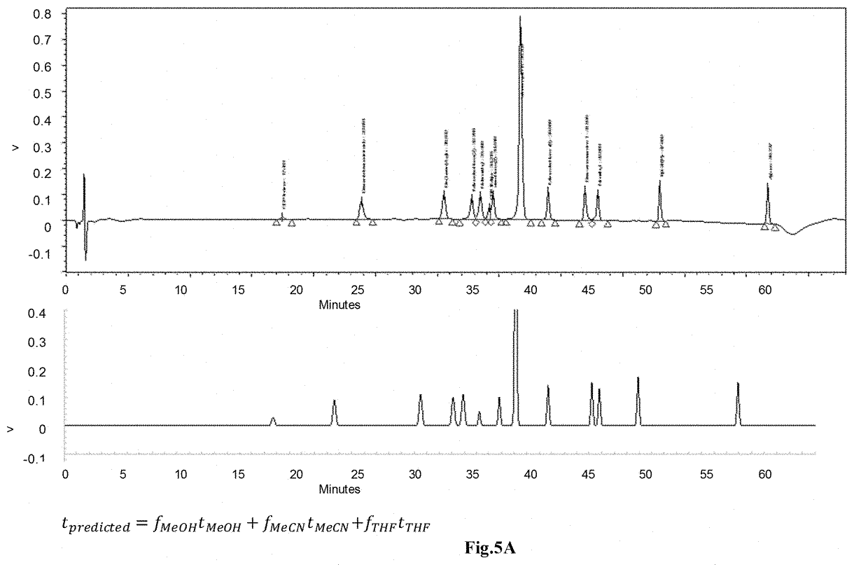

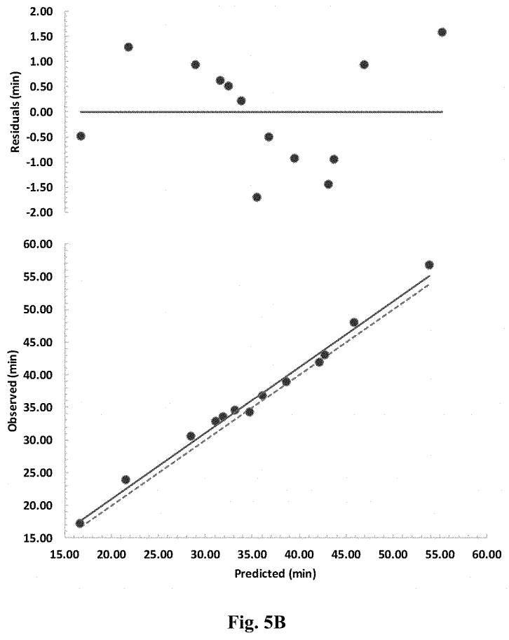

FIG. 5A compares the observed (upper) to predicted (lower) separation using a ternary mobile phase comprised of pH 4.5 buffer (sodium acetate/acetic acid)/acetonitrile/THF. Compared is an optimized separation of vancomycin and 12 known impurities, showing a computer simulation of the predicted optimum separation (lower chromatogram) and the observed chromatogram (upper). The retention times are generally longer in the observed chromatogram than predicted. Baseline separation of all peaks was observed except between CDP-1Major and cis-chloro-(2)-vancomycin. FIG. 5B shows the excellent statistical agreement between the observed and predicted retention times of vancomycin and 12 known impurities.

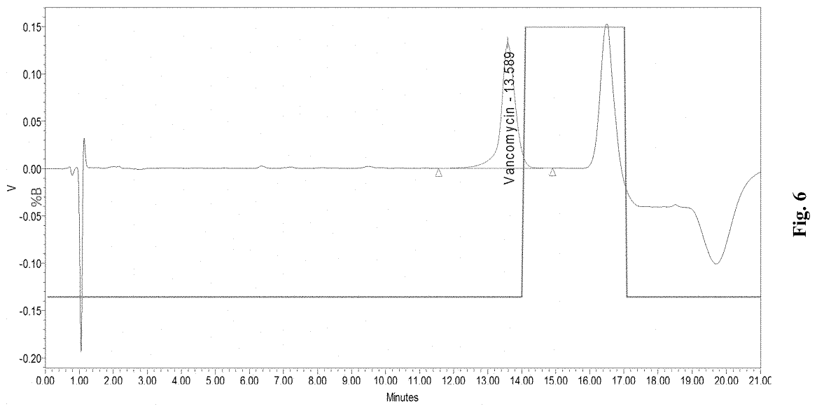

FIG. 6 shows a chromatogram of a simplified ternary system used for an assay method, which requires short retention times due to a large number of samples that need to be tested compared to an impurity analysis. The assay method is based on modifying the method developed for impurities that uses a ternary system of buffer with THF and acetonitrile organic solvents. A simple step gradient is employed, wherein a 80:20 mixture of Mobile Phase A:Mobile Phase B is run for 14 minutes. After elution of vancomycin, 100% Mobile Phase B is run for 3 minutes to wash the column, followed by more of the 80:20 mixture to re-set the column for the next run. The second positive and the third negative peak after vancomycin are baseline changes arising from the step gradient. The operating conditions for the assay of vancomycin are shown below.

TABLE 3
Operating Conditions for the Assay Method

| Guard Column | None | |
|---|---|---|
| Autosampler | 4° C. | |
| Temperature | | |
| Column | ACE Excel 2 PFP-C18, 2.1 mm × 100 mm, 2 μm | |
| Column Temperature | 40° C. | |
| Flow Rate | 0.4 mL/min | |
| Injection Volume | 20 μL | |
| UV Detection | 230 nm | |
| Wavelength | | |
| Mobile Phase A | Sodium Acetate Buffer, pH 4.1 | |
| Mobile Phase B | 2.19:82.0:15.6 THF/Sodium Acetate Buffer/ | |
| | Acetonitrile | |
| Run time | 21 minutes | |
| Gradient | Time (min) | % B |
| | 0 | 20 |
| | 14 | 20 |
| | 14.1 | 100 |
| | 17 | 100 |
| | 17.1 | 20 |
| | 21 | 20 |

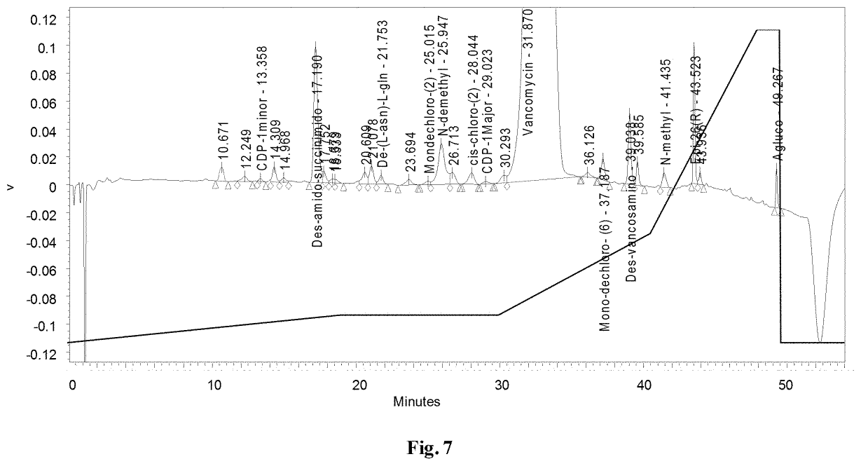

FIG. 7 shows a chromatogram of an optimized impurities method wherein a more complex gradient profile is run, shown superimposed on the chromatogram. It shows separation of twelve known impurities and several additional unidentified impurities from vancomycin. The run time is significantly longer than the assay method. The parameters of the impurity method are summarized below.

TABLE 4
Operating Conditions for the Impurity Method

| Guard Column | None | |
|---|---|---|
| Autosampler | 4° C. | |
| Temperature | | |
| Column | ACE Excel 2 PFP-C18, 2.1 mm × 100 mm, 2 μm | |
| Column Temperature | 40° C. | |
| Flow Rate | 0.4 mL/min | |
| Injection Volume | 20 μL | |
| UV Detection | 230 nm | |
| Wavelength | | |
| Mobile Phase A | Sodium Acetate Buffer, pH 4.10 | |
| Mobile Phase B | 2.19:82.0:15.6 THF/Sodium Acetate Buffer/ | |
| | Acetonitrile | |
| Run time | 54 minutes | |
| Gradient | Time (min) | % B |
| | 0 | 8 |
| | 19 | 16 |
| | 30 | 16 |
| | 40.5 | 40 |
| | 48 | 100 |
| | 49.5 | 100 |
| | 49.6 | 8 |

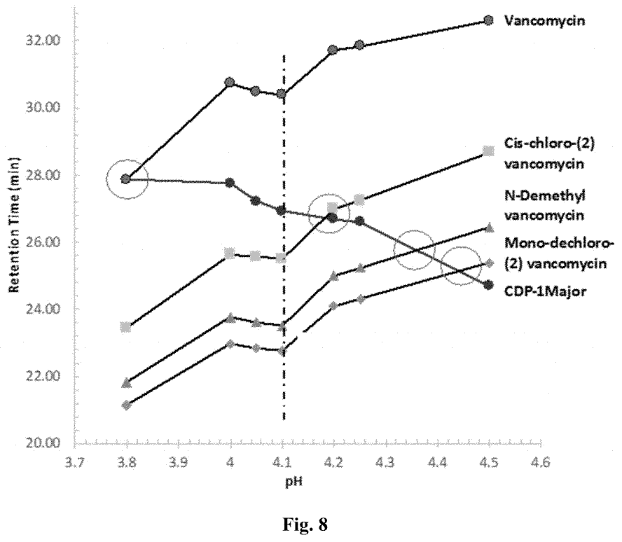

FIG. 8 shows the effect of pH on the separation of vancomycin and four difficult to separate impurities. The graphs in FIG. 8 show how an additional acidic pKa (approximately 4.5) for des-(amido)-aspartyl-vancomycin (also known as CDP-1 Major) due to the replacement of the neutral amino acid glutamine with iso-aspartic acid, can affect the separation. It shows that reproducible separation requires tight control of pH to avoid coelution of compounds. Notably, depending on the pH, the impurity CDP-1 Major coeluted with any of four other compounds when the pH ranged from 3.8 to 4.5. The best separation required control of pH±0.02. The target pH changed gradually over time and required periodic re-setting of conditions.

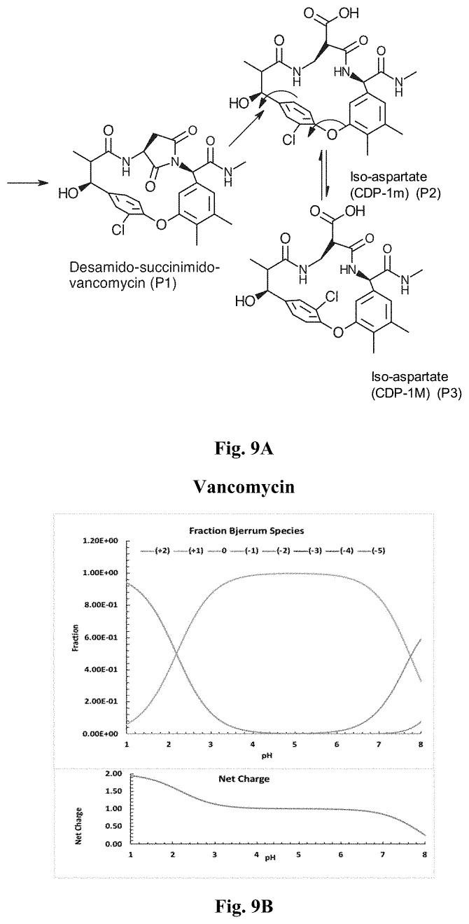

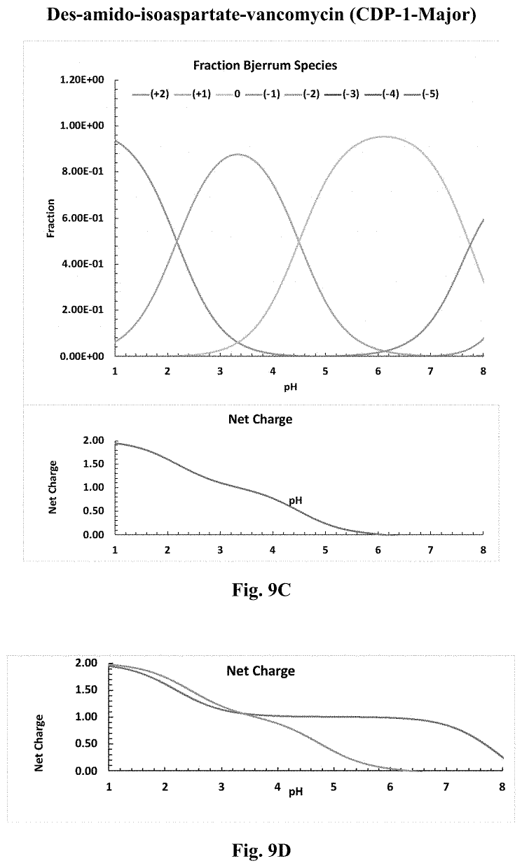

FIGS. 9A-9D show the effect of pH on the net charge of vancomycin and des-amido-isoaspartate vancomycin, showing the effect of an additional acidic pKa (approximately 4.5) due to the replacement of the neutral amino acid, glutamine with iso-aspartic acid. FIG. 9A shows a portion of the degradation pathways of vancomycin that leads to des-amido-aspartyl vancomycin, which exists as two different conformers, CD-1 Major and CD-1 Minor. FIG. 9B shows the effects of pH on the ionized form and net charge of vancomycin in the pH range of 1 to 8. FIG. 9C shows the effects of pH on the ionized form and net charge of des-amido-isoaspartate-vancomycin in the pH range of 1 to 8. FIG. 9D compares the effects of pH on the net charge of vancomycin and des-amido-isoaspartate-vancomycin in the pH range of 1 to 8.

The dissociation constants of vancomycin and des-amido-isoaspartyl-vancomycin (CDP-1 Minor and CDP-1 Major) are assumed to have very similar pKa values and illustrate why the pH needs to be very carefully controlled. While vancomycin shows a plateau in net charge between about 3 and 6, des-amido-iso-aspartyl-vancomycin (CDP-1 Minor and CDP-1 Major) does not show a similar plateau due to its additional ionization site at about 4.5. The net charge of the two compounds intersects at a pH of about 3.6. This narrows considerably the range of pH where an effective separation can be achieved.

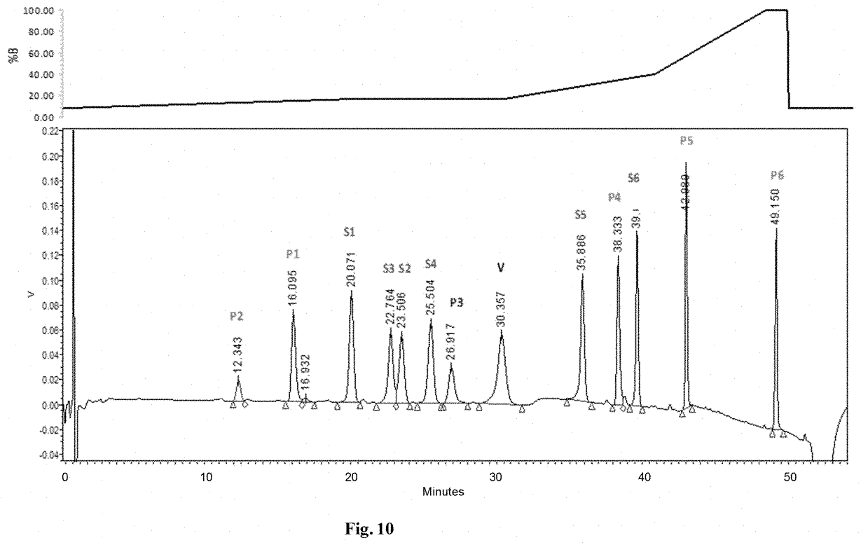

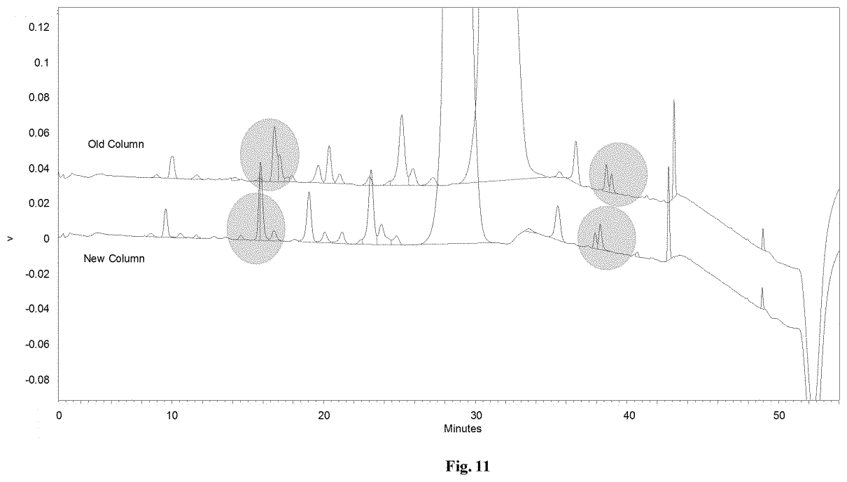

The first optimized analytical method utilizes reversed phase HPLC with an ACE Excel 2 C18-PFP column with the specific parameters shown in Table 4. FIG. 10 shows the gradient profile and a chromatogram of the separation of vancomycin and 12 known impurities on a 10-cm PFP-C18 column. The method was validated and used for routine sample analysis. More than one batch of columns, 10 cm in length, gave the same separations. This method worked for a considerable period of time without change in chromatography. After more than a year of successful application of the method with several PFP-C18 columns, subtle changes in separation of critical pairs of peaks with a new PFP-C18 column were observed. The separation achieved with the original columns could not be reproduced with any new PFP-C18 column. In general, the retention times were longer using the old column and some peaks eluted in a different order, compared to the new column (FIG. 11). Revision of the mobile phase composition on the new columns to achieve the required separation was unsuccessful. This required a complete rework of the method, armed with previous experience.

We also wished to develop a single method for the analysis of vancomycin impurities in both AeroVanc and in vancomycin hydrochloride drug substance, the active pharmaceutical ingredient (API) in AeroVanc. The previous method was acceptable for AeroVanc but was not acceptable for drugs substance analysis, which requires the analysis of all known and unknown impurities in vancomycin hydrochloride, including separation of several pairs of peaks not well separated by the original method developed for AeroVanc.

Preliminary studies indicated that a different approach was required. The same stationary phase (PFP C18) was used due to its ability to separate closely structures, but a longer 15-cm column was required to give greater separating power.

We chose to investigate a combination of pH gradient and solvent gradient. Ion-exchange chromatography commonly uses pH gradients. Solvent gradients are standard in reversed phase chromatography. A combined pH/solvent gradient is an unconventional approach in reversed phase chromatography. The combination of solvent gradient and pH gradient was achieved by mixing two different mobile phase compositions having different pH values and different solvent strengths to provide a combined mobile phase in which pH and solvent strength could be varied depending on the ratio of the two mobile phase compositions.

A second even more improved method was developed using a more complex DOE (DOE-2), exploring additional variabilities such as flow rate, gradient shape, and temperature. A second design of experiment (DOE-2), more extensive than the first DOE-1 used to develop the original method, examined the effects of acetonitrile, THF, buffer pH, column length, temperature and gradient profile on the separation. The second design of experiments (DOE-2) study suggested that isocratic (constant solvent strength) was required to separate peaks eluting before vancomycin and a gradient (increasing solvent strength) was required to separate peaks eluting after vancomycin. This dramatically increased the number of potential variables to be optimized. Additional studies indicated that a different buffer pH was required for the isocratic regions compared with the buffer pH in the gradient region, leading to a concomitant pH gradient and a solvent gradient as the overall strength of the mobile phase was increased.

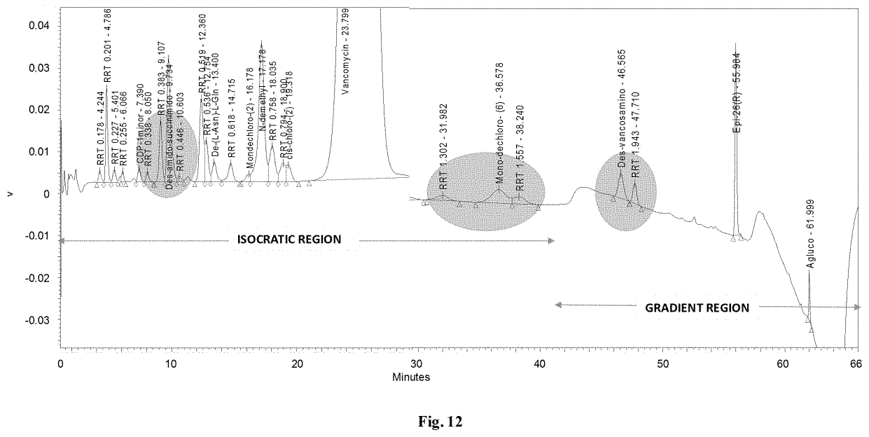

These studies predicted an improved method could be obtained with an initial isocratic region (constant mobile phase conditions) to separate the early eluting peaks followed by a gradient region to separate the late eluting peaks. Initial experiments also showed a longer 15-cm column compared with the 10-cm column used previously was needed to separate all the peaks. The two regions of the chromatography were optimized separately, using an isocratic region for early peak resolution and a gradient region for peaks eluting after vancomycin. FIG. 12 shows a combined chromatogram showing an isocratic separation and a gradient separation obtained at different times. Optimum separation of the late eluting peaks requires a difference in pH and solvent composition of the second mobile phase component compared to the pH and solvent composition of first mobile phase component. The challenge remained to develop a single method that merged both the isocratic region and the gradient region to achieve successful separations of difficult pairs of peaks at both early and late portions of the chromatogram. The results of these initial scouting experiments suggested that this approach could work.

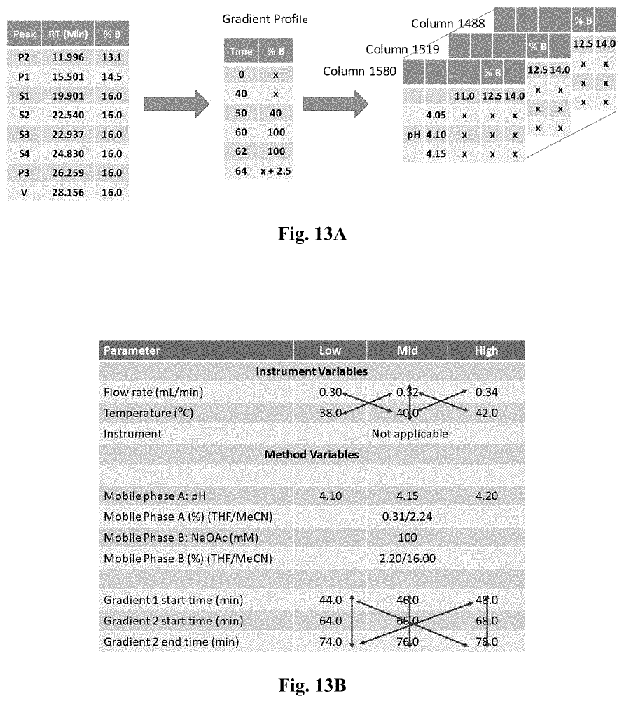

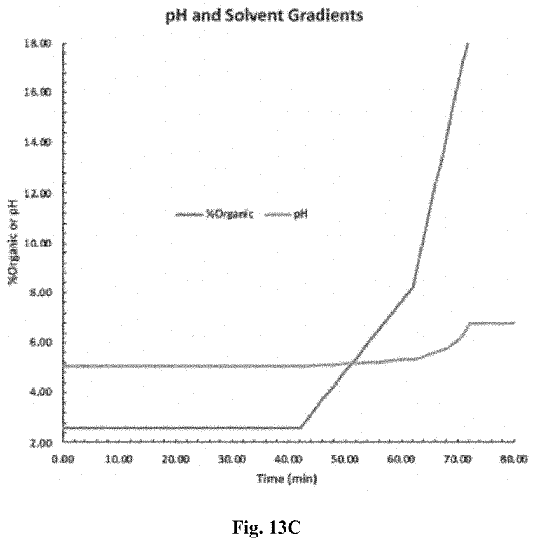

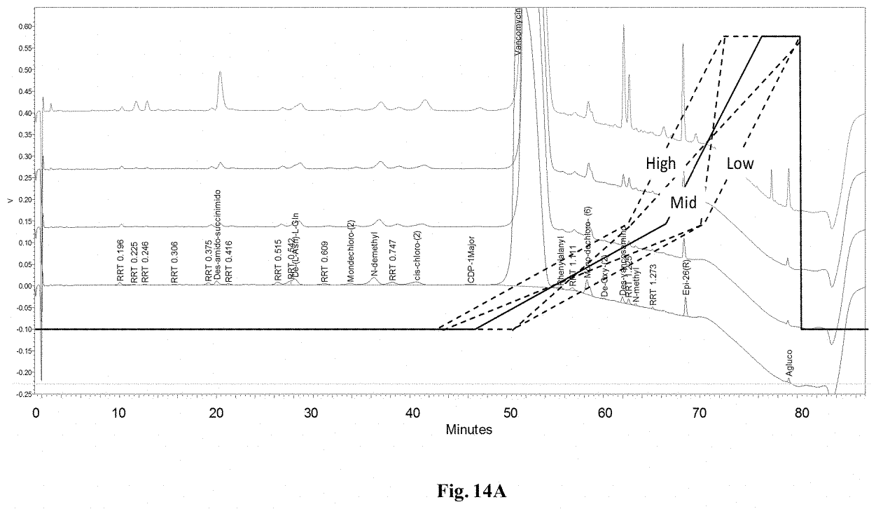

FIG. 13A shows the Design of Experiment (DOE-2) conducted to demonstrate the robustness of the method in isocratic region of the chromatogram on three 15-cm PFP-C18 columns, using three pH values (4.05, 4.10 and 4.15) and three concentrations of organic solvent (11.0% B, 12.5% B and 14.0% B, where B represents the second mobile phase component). In these experiments the gradient profile was held constant.

FIG. 13B shows the Design of Experiment (DOE-2) conducted to demonstrate the robustness of the method in gradient region of the chromatogram on a single 15-cm PFP-C18 column using different gradient slopes, different pH values and different temperatures. These variables are in the range we have defined for validation of the method. This range is intentionally limited because validation is designed to define the range within which intentional small changes in instrument settings can be done without affecting the separation. A flow rate in the range of 0.30 mL/min to 0.34 mL/min is preferred (FIG. 13B). A column temperature in the range of 38.0° C. to 42.0° C. is preferred (FIG. 13B). A composition of 0.31% THF (by weight), and 2.24% acetonitrile (by weight) is preferred for the first mobile phase component (FIG. 13B). A composition of 2.20% THF (by weight), and 16.00% acetonitrile (by weight) is preferred for the second mobile phase component (FIG. 13B). A pH in the range of 4.10 to 4.20 is optimum for the separation of the early peaks in the isocratic region. A higher pH achieved with no acetic acid and 25 mM (pH 8.5), 75 mM (8.7), 100 mM (8.8) or 125 mM (8.8) sodium acetate is required for the second mobile phase. The separation of the late eluting peaks requires a pH gradient as well as an organic solvent gradient for (FIG. 13C). This difference in pH values and solvent can be achieved by gradiently combining the two mobile phase components. Mixing the first mobile phase component with the second mobile phase component results in a continuous but non-linear change in pH of the combined mobile phase, together with two linear organic-solvent gradients of increasing slope, passing though the column.

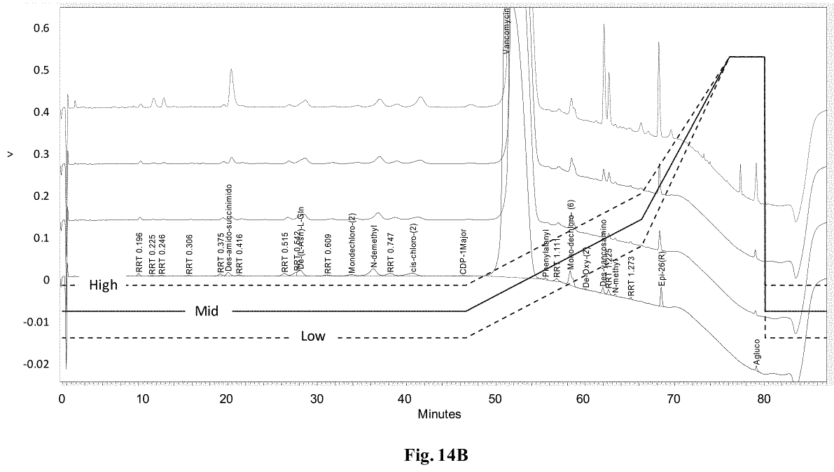

The results in FIGS. 14 to 21 show how variation of the conditions, described in FIG. 13B, was investigated to find the “sweet spots” in the isocratic region (FIG. 14B) and gradient region (FIG. 14A) of the separation.

FIG. 14A shows the design space for the gradient region of the chromatogram for the separating the peaks eluting after vancomycin. FIG. 14A compares three different gradient profiles and their effects on separation of the peaks. In FIG. 14B, the lines labeled “High”, “Mid” and “Low” correspond to the gradient times indicated in the table in FIG. 13B. For example, the “High” gradient 1 starts at 44 minutes and the “High” gradient 2 starts at 68 minutes and ends at 78 minutes. FIG. 14B shows the design space for the isocratic region of the chromatogram for the separating the peaks eluting before vancomycin. It compares three different solvent concentrations and pH and their effects on the separation. The “High”, “Mid” and “Low” correspond to the pH values indicated in the table in FIG. 13B.

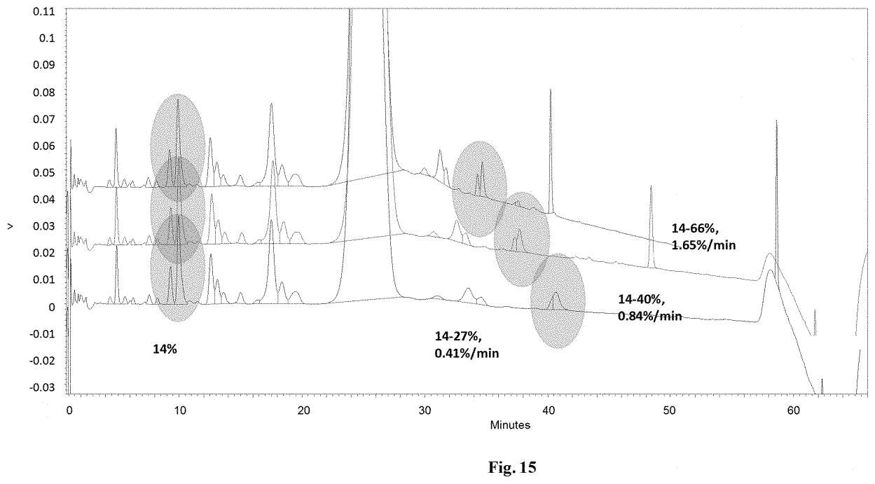

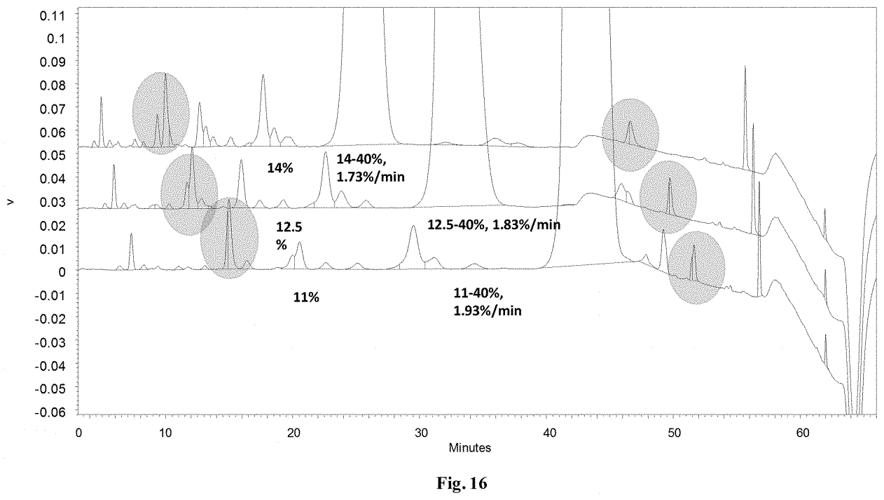

As examples, FIG. 15 and FIG. 16 show some of the results of changing the conditions for the isocratic region and the gradient region of the chromatograms obtained on a 10-cm column. FIG. 15 shows the effect of changing the gradient profile on a 10-cm column, showing that the highlighted peaks can be separated by using a “high” pH gradient. FIG. 16 shows the effects of changing both the total solvent concentration in the first mobile phase component and the gradient profile for the second mobile phase component on the overall separation of the peaks eluting before and after vancomycin on the 10-cm column, showing incomplete resolution of desamido-succinimido vancomycin and two unknown peaks (highlighted peaks eluting before vancomycin) and the incomplete resolution of the desvancosamino-vancomycin from an unknown peak (eluting after vancomycin).

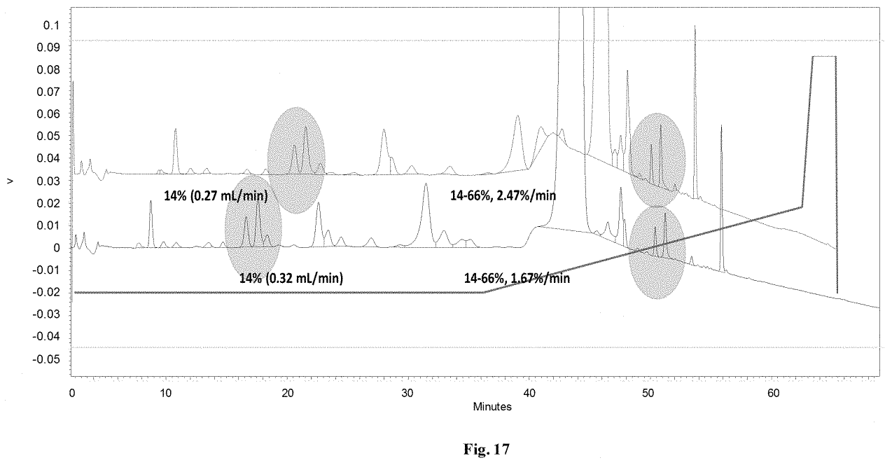

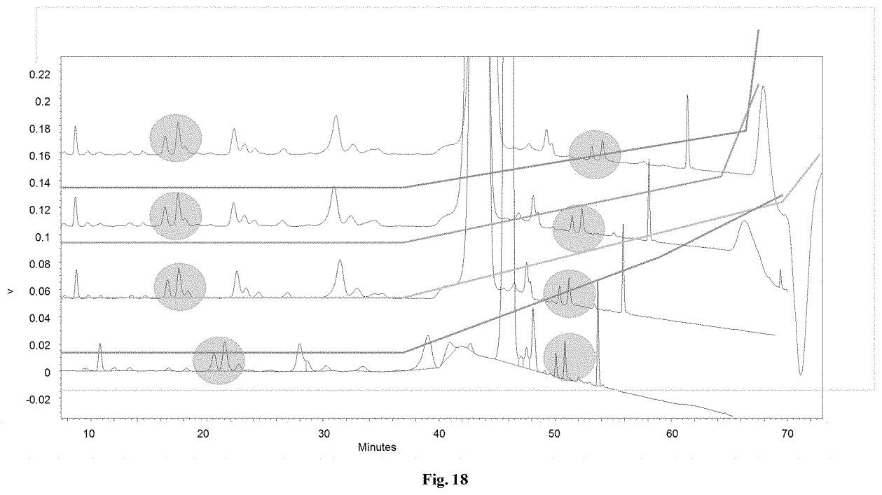

Both FIG. 15 and FIG. 16 show that the separation of certain pairs of peaks were unresolved on the 10-cm column. For example, desamido-succinimido vancomycin and two unknown peaks eluting before vancomycin, and desvancosamino vancomycin and an unknown peak eluting after vancomycin were only partially resolved on the 10-cm column (FIG. 15 and FIG. 16). The problem of incomplete resolution of these sets of peaks was solved by replacing the 10-cm column with a longer 15-cm column containing the same C18-PFP packing material (FIGS. 17 and 18). Replacement of the 10-cm column with a 15-cm column produced longer retention times for the peaks in the isocratic region but had minimal effect on the retention times of the peaks eluted in the gradient region. The effect of increasing the column length on analysis time was off-set by increasing the flow rate without adversely affecting the overall separation (FIG. 17 and Table 5). FIG. 18 shows the effect of flow rate and gradient shape on separation and analysis time on a 15-cm column. Shorter analysis time was feasible with increased flow rate, up to the maximum pressure limit of the instrument used (36 mPa).

TABLE 5
Isocratic and Gradient Conditions on the 15-cm Column

| Column | 1619 | Column | 1619 |
|---|---|---|---|
| pH(calc) | 4.05 | pH(calc) | 4.05 |
| pH(exp) | 4.04 | pH(exp) | 4.04 |
| | 0.27 mL/minute | | 0.32 mL/minute |
| Minutes | % of Mobile Phase B | Minutes | % of Mobile Phase B |
| 0 | 14 | 0 | 14 |
| 36 | 14 | 36 | 14 |
| 57 | 66 | 67 | 66 |
| 67 | 100 | 71 | 100 |
| 69 | 100 | 73 | 100 |
| 69.1 | 100 | 73.1 | 100 |

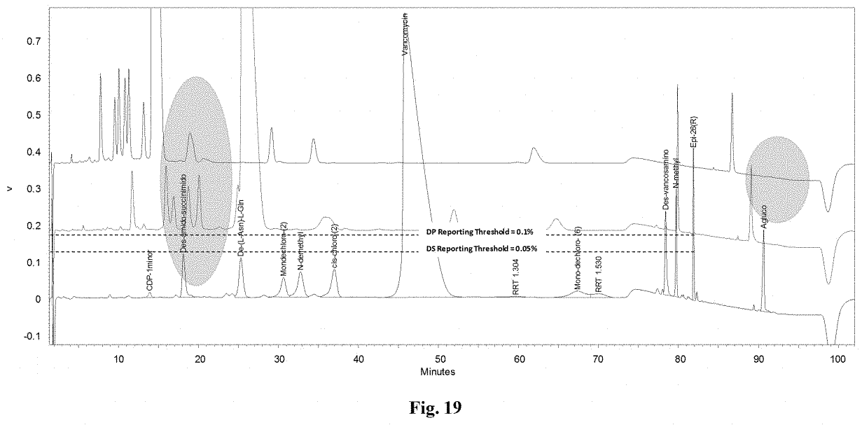

FIG. 19 shows the effect of column temperature on separation and analysis time on the 15 cm column (from top to bottom, the temperatures are 70° C., 55° C. and 40° C.). FIG. 19 shows marked decrease in retention times and reduction in resolution of adjacent peaks when increasing the temperature from 40° C. to 55° C. and then increasing the temperature from 55° C. to 70° C.

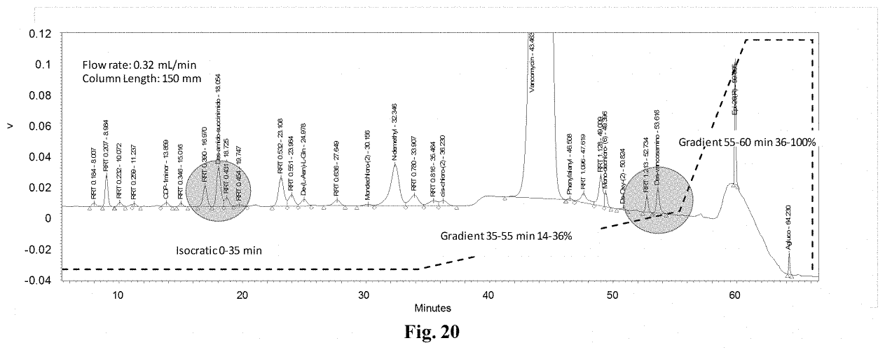

FIG. 20 shows a chromatogram of the optimized separation of known and unknown impurities of vancomycin in AeroVanc in which the more difficult resolutions are achieved.

The improved HPLC method was validated according the Q2(R1) guideline of the International Council on the Harmonization (ICH) of the Technical Requirements for Pharmaceuticals for Human Use (ICH Q2(R1)) and shown to be fit for the purpose.

The improved method can accurately and precisely measure the concentration of each impurity within the range 0.05% to 10.0% relative to vancomycin.

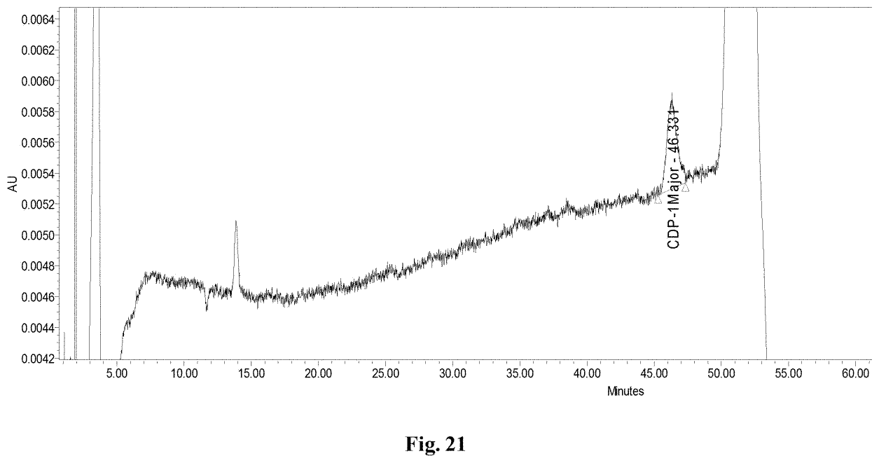

FIG. 21 shows a chromatogram of CDP-1 Minor and CDP-1 Major at a combined concentration of 0.05% relative to vancomycin, illustrating the sensitivity of the method.

HPLC Instruments

The retention time of vancomycin and the impurities vary among instruments. Therefore, the gradient start times may be adjusted, within the ranges specified in the method, to achieve the required retention times and to meet the system suitability criteria. The start time of the first gradient (ST₁) (Table 5) is defined in initial instrument set up-conditions. The gradient slopes of each region are constant defined by the value of ST₂, ST₃, ST₄, ST₅ and ST₆ in Table 6.

HPLC Columns

The retention times of the vancomycin and the impurities can increase very gradually over time with repeated use for analysis of routine samples, and the selectivity of the column changes subtly over time as the column ages. The method is more flexible than the original method and conditions can be re-adjusted (Table 6 below) when using one column for a period of time or when a new column is used. The gradient, acetic acid concentration and sodium acetate concentration in the mobile phase can be adjusted to achieve the required retention times and to meet the system suitability criteria. Other factors related to the column itself can influence the separations. For example, changes in column manufacture may slightly change their composition, which can have a significant effect on separations.

TABLE 6
Operating Conditions for Improved Method for
Impurities in Vancomycin Hydrochloride (Drug
Substance) and AeroVanc (Drug Product)

| Guard Column | None | |
|---|---|---|
| Autosampler | 4° C. | |
| Temperature | | |
| Column | ACE Excel 2 C18-PFP, 2.1 mm × 150 mm, 2 μm | |
| Column | 40° C. | |
| Temperature | | |
| Flow Rate | 0.32 mL/min | |
| Injection Volume | 20 μL | |
| UV Detection | 230 nm | |
| Wavelength | | |
| Mobile Phase A | THF/Buffer A/Acetonitrile 2.5/979.9/17.3 (w/w/w) | |
| Mobile Phase B | THF/125 mM Sodium Acetate/Acetonitrile 19.0/ | |
| | 820.5/120.7 (w/w/w) | |
| Run time | ~85 minutes | |
| Gradient | Time (min) | % B |
| | 0 | 0 |
| | ST₁ | 0 |
| | ST₂ = ST₁ + 20 | 35 |
| | ST₃ = ST₂ + 10 | 97 |
| | ST₄ = ST₃ + 4 | 97 |
| | ST₅ = ST₄ + 0.1 | 0 |
| | ST₆ = ST₁ + 6.9 | 0 |

Autosampler Needle Rinse Instructions

| | |
|---|---|
| Rinse Solvent | Water |
| Rinse Volume | 200 μL |
| Needle Stroke | 52 mm |
| Sampling Speed | 5.0 μL/sec |
| Rinse Speed | 35 μL/sec |
| Rinse Dip Time | 10 sec |
| Purge Time | 1.0 min |
| Rinse Mode | Before and after aspiration |

Procedures

Reference Standards

Vancomycin HCl and its impurities are light sensitive and hygroscopic.

Reference standards are handled in a humidity chamber or controlled humidity room that has been purged with nitrogen or low relative humidity air prior to use and confirmed to have a relative humidity less than 10% (as measured with the Vaisala Hygrometer, or a similar device).

The water content of the vancomycin reference standard is re-measured by a separate technique each time the standard solutions are prepared.

All standard and sample solutions are prepared in Class A volumetric glassware, stored at 2-8° C. and protected from light by wrapping in aluminum foil. HPLC vials containing the solutions are protected from light by covering the HPLC vials with foil on the bench-top prior to transferring the vials to the autosampler.

Retention Time Markers

Vancomycin has 12 impurities that are known to be present in samples of vancomycin hydrochloride and in vancomycin hydrochloride inhalation powder and capsules, as well as a number of unidentified impurities.

Six of the 12 known impurities are potential degradation products of vancomycin:

Des-amido-isoaspartyl vancomycin (minor isomer, CDP-1 Minor) (EP Impurity B)

Des-amido-isoaspartyl vancomycin (major isomer, CDP-1 Major) (EP Impurity B)

Des-amido-succinimido vancomycin

Des-vancosamino vancomycin (EP Impurity D)

Epi-26(R) vancomycin

Agluco vancomycin (EP Impurity C)

The other six impurities arise from the drug substance:

Cis-chloro-(2) vancomycin

N-demethyl vancomycin (EP Impurity A)

Mono-dechloro-(2) vancomycin

De-(L-Asn)-L-Gln vancomycin

N-methyl vancomycin

Mono-dechloro-(6) vancomycin

Mono-dechloro-(6) vancomycin sometimes elutes as a pair of poorly resolved peaks in all the samples as well as in the reference standard of mono-dechloro-(6) vancomycin, suggesting the compound is a mixture of two compounds. The two peaks should be integrated together as one peak and reported as mono-dechloro-(6) vancomycin.

The peaks for vancomycin and five of the known impurities are located by the injection of reference standards as follows:

Des-amido-iso-aspartyl vancomycin (a mixture of CDP-1Minor and CDP-1Major in the approximate ratio of 3:7)

N-demethyl vancomycin

Des-vancosamino vancomycin

Agluco vancomycin

The expected retention times of the other known impurities are calculated from the known relative retention times as described below. Unidentified (unknown) impurities may be present in samples of vancomycin formulations. The retention times of the expected unidentified impurities are calculated from the retention times of the reference standards as described below.

### Examples

General Procedures for chromatographic separations are described below.

Materials and Equipment

Equipment

Shimadzu HPLC System, or equivalent, comprising the following components or equivalents.

LC-20AD, binary HPLC pumps with 180 μL mixer

SIL-20AC, autosampler

CTO-20A, column heater

DGU-20A, inline degasser

SPD-20A, UV/Vis detector or SPD-M10A diode array detector

CBM-20A communications Bus Module

Computer or other suitable data collection system

ACE Excel 2 C18-PFP HPLC column (part number EXL-1010-1502U) having an internal diameter of 2.1 mm, length of 150 mm and particle size of 2 μm.

Analytical balance: AND HM-202 or equivalent

Sartorius ME5 Semi-micro analytical balance or equivalent

Top-loading Balance, Mettler Toledo PB4002-S or equivalent

Humidity chamber (Nitrogen Glove Box)

pH Meter, Fisher Scientific AR25 or equivalent

Refrigerator capable of maintaining temperature of 2-8° C.

Freezer capable of maintaining temperature of −20° C.

Vaisala hygrometer or other device capable of measuring relative humidity below 10%

Gilson positive displacement adjustable micropipets, MR-1000 and MR-250 or equivalent.

Class A volumetric flasks having volumes of 2, 5, 25 and 250 mL

Class A 5.0 mL volumetric pipets

Scintillation vials, clear glass, 20 mL

Pointed Glass Rod, EMD part number 1.09998 or equivalent

Chemicals and Reagents

Vancomycin hydrochloride API reference standard

Des-amido-isoaspartate vancomycin reference standard

Agluco vancomycin reference standard

N-demethyl vancomycin reference standard

Des-vancosamino vancomycin reference standard

Vancomycin hydrochloride inhalation bulk powder (AeroVanc Lot 195A01-NJ00011-S3)

Water, HPLC grade

Acetonitrile, HPLC grade

Tetrahydrofuran, 99.9+%, anhydrous, inhibitor-free

Sodium Acetate 3M Solution, BioUltra Grade (Sigma P/N 71196-100ML or equivalent)

Acetic Acid, Ampules to make 500 ml of 1 N Acetic Acid, (EMD Titrisol part number 1.09951.0001 or equivalent)

Sodium phosphate monobasic, monohydrate, HPLC Grade or equivalent

Phosphoric acid, 85%, ACS grade or equivalent

Solutions were prepared according to procedures described below. Quantities were scaled as needed to prepare different volumes of the solutions.

1N Acetic Acid Solution

Rinse a 500 ml volumetric flask well with water. Add approximately 250 ml of water to the flask. Pierce one ampoule of Titrisol acetic acid in the flask using a pointed glass rod and rinse out the ampoule contents and the rod with sufficient amounts of water to transfer the acid completely to the flask. Dilute the flask to volume with water and mix well. Store at room temperature and assign an expiration date of seven days from the date of preparation.

Buffer A: Sodium Acetate Buffer, pH 4.10

Rinse a 2 L volumetric flask well with water and add approximately 200 ml of water to the flask. Dry the outside of the flask completely and tare the flask on the top-loading balance. Add 150.00 g±0.05 g of 1 N Acetic Acid Solution to the flask. Record the weight of 1 N Acetic Acid Solution in grams to two decimal places. Calculate the target weight of 3 M sodium acetate solution in grams from the certificate of analysis using the following formula

$$W = \frac{{M}_{T} \times 1000 \times 1.112 \times V}{M}$$

Where:
W=Target weight of 3 M sodium acetate solution (in g)
M_{T}=Target molar concentration of sodium acetate solution (0.0251 M)
M=Exact molarity of 3 M sodium acetate solution (from supplier certificate of analysis)
1000=Conversion from L to mL
1.112=Density of 3 M sodium acetate solution (g/mL)
V=Final Volume of Buffer A

Sodium Acetate Solution, 125 mM

Rinse a 1 L volumetric flask well with water and add approximately 200 ml of water to the flask. Dry the outside of the flask completely and tare the flask on the top-loading balance. Calculate the target weight of 3 M sodium acetate solution in grams from the certificate of analysis using the formula

$$W = \frac{{M}_{T} \times 1000 \times 1.112 \times V}{M}$$

Where:
W=Target weight of 3 M sodium acetate solution (in g)
M_{T}=Target molar concentration of sodium acetate solution (0.125M)
M=Exact molarity of 3 M sodium acetate solution (from supplier certificate of analysis)
1000=Conversion from L to mL
1.112=Density of 3 M sodium acetate solution (g/mL)
V=Final Volume of Sodium Acetate Solution

Add the calculated target weight W±0.05 g of 3 M sodium acetate solution to the flask. Record the target weight and experimental weight of 3 M sodium acetate solution in grams to two decimal places. Dilute the solution to volume using water and mix well by inverting at least twenty times. Store at room temperature and assign an expiration date of 7 days from the date of preparation.

THF Stock Solution: THF/Buffer A, 1:4 (w/w), to Prepare 250 Milliliters

Rinse a 0.5 L mobile phase bottle well with water and drain completely. Dry the outside of the bottle well. Tare the 0.5 L mobile phase bottle on the top-loading balance. Add 200.00 g±0.05 g of Buffer A into the 0.5 L glass bottle. Tare the balance again and add 50.0 g±0.05 g THF into the 0.5 L glass bottle. Cap and mix the solution thoroughly using a stir-bar. Store at room temperature and assign an expiration date of 7 days from the date of preparation.

Mobile Phase A: THF/Buffer A/Acetonitrile 2.5/979.9/17.3 (w/w/w), to Prepare 1.8 Liters

Rinse a 2 L mobile phase bottle well with water and drain completely. Dry the outside of the bottle well. Tare the 2 L mobile phase bottle on the top-loading balance. Add 1745.64 g±0.05 g of Buffer A into the 2 L glass bottle. Tare the balance again and add 22.68 g±0.05 g THF Stock Solution into the 2 L glass bottle. Tare the balance again and measure 31.24±0.05 g acetonitrile into the 2 L glass bottle. Cap and mix the solution thoroughly using a magnetic stir-bar. Store at room temperature and assign an expiration date of 7 days from the date of preparation.

Mobile Phase B: THF/125 mM Sodium Acetate/Acetonitrile 19.0/820.5/120.7 (w/w/w), to Prepare 2 Liters

Rinse a 1 L mobile phase bottle well with water and drain completely. Dry the outside of the bottle well. Tare the 1 L mobile phase bottle on the top-loading balance. Add 820.50 g±0.05 g of 125 mM Sodium Acetate Solution into the 1 L glass bottle. Tare the balance again and add 19.00 g±0.05 g THF into the 1-liter glass bottle. Tare the balance again and add 120.70±0.05 g acetonitrile into the 1 L glass bottle. Cap and mix the solution thoroughly using a magnetic stir-bar. Store at room temperature and assign an expiration date of 7 days from the date of preparation.

### Diluent 1—25 mM Sodium Phosphate Buffer, pH 3.2 (to Prepare 1 L)

Weigh 3.45 g of sodium phosphate, monobasic, monohydrate and add to the 1 L glass bottle. Add 1000 mL water to the 1 L glass bottle. Stir until dissolved. Adjust the pH of the solution to 3.2 using 85% phosphoric acid while stirring. Store at room temperature and assign an expiration date of 30 days from the date of preparation.

### Diluent 2—50:50 25 mM Sodium Phosphate Buffer, pH 3.2/Acetonitrile to Prepare 500 mL

Measure 250 ml Diluent 1 into a 500 mL glass bottle. Measure 250 ml acetonitrile and add to the 500 mL glass bottle. Mix thoroughly. Store at room temperature and assign an expiration date of 30 days from the date of preparation.

### Standard Solutions

### Vancomycin Standard Solutions—Standard A and Standard B

The nominal concentration of standard solution is approximately 90 μg/mL without correction for water and purity. Quantities may be scaled as appropriate to prepare different volumes of the solutions.

Determine the water content of the vancomycin HCl reference standard by Karl-Fisher titration and record the value. Obtain two moisture values, one before weighing the standards and one after weighing the standards. Use the mean value of the moisture value in the calculations. Accurately weigh approximately 22.50±1.00 mg of vancomycin HCl reference material and record the weight in mg to two decimal places. Transfer to a 250 mL volumetric flask with aid of Diluent 1. Dissolve and adjust to 250 mL with Diluent 1. Mix well and label as Standard A.

Prepare a second standard solution by weighing approximately 22.50±1.00 mg of vancomycin HCl reference material and recording the weight in mg to two decimal places. Transfer to a 250 mL volumetric flask with aid of Diluent 1. Dissolve and adjust to 250 mL with Diluent 1. Label the second standard solution as Standard B. Store both solutions at 2-8° C., protect from light and assign an expiration date of 14 days from the date of preparation.

### Vancomycin Impurities Stock Solutions

Des-Amido-Isoaspartate Vancomycin (approximately 500 μg/mL)

Accurately weigh 1.000 to 1.200 mg des-amido-isoaspartate vancomycin using the microbalance. Record the weight in mg to three decimal places. Transfer to a 2 mL volumetric flask, with the aid of Diluent 2. Mix thoroughly until dissolved. Adjust the volume to 2 mL with Diluent 2. Mix thoroughly. Store at −20° C. until needed.

N-Demethyl Vancomycin (Approximately 500 μg/mL)

Accurately weigh 1.000 to 1.200 mg N-demethyl vancomycin using the microbalance. Record the weight in mg to three decimal places. Transfer to a 2 mL volumetric flask, with the aid Diluent 1. Mix thoroughly until dissolved. Adjust the volume to 2 mL with Diluent 1. Mix thoroughly. Store at −20° C. until needed.

Des-Vancosamino Vancomycin (Approximately 500 μg/mL)

Accurately weigh 1.000 to 1.200 mg desvancosamino-vancomycin using the microbalance. Record the weight in mg to three decimal places. Transfer to a 2 mL volumetric flask, with the aid of Diluent 2. Mix thoroughly until dissolved. Adjust the volume to 2 mL with Diluent 2. Mix thoroughly. Store at −20° C. until needed.

Agluco Vancomycin (Approximately 500 μg/mL)

Accurately weigh 1.000 to 1.200 mg agluco vancomycin using the microbalance. Record the weight in mg to three decimal places. Transfer to a 2 mL volumetric flask, with the aid of Diluent 2. Mix thoroughly until dissolved. Adjust the volume to 2 mL with Diluent 2. Mix thoroughly. Store at −20° C. until needed.

### Impurity Marker Solution

Prepare the Impurity Marker Solution containing the following mixture:

Des-amido-isoaspartate vancomycin approximately 45 μg/mL (total CDP-1Minor+CDP-1Major), Agluco vancomycin approximately 45 μg/mL, Des-vancosamino vancomycin approximately 45 μg/mL, N-demethyl vancomycin approximately 45 μg/mL, Vancomycin approximately 900 μg/ml.

Accurately weigh 4.500±0.500 mg vancomycin HCl reference standard on the microbalance. Transfer to a 5 mL volumetric flask with the aid of Diluent 1. Mix thoroughly until dissolved. Pipet 450 μL des-amido-isoaspartate vancomycin, approximately 500 μg/mL, into the 5 mL volumetric flask. Pipet 450 μL N-demethyl vancomycin, approximately 500 μg/mL into the 5 mL volumetric flask. Pipet 450 μL des-vancosamino vancomycin approximately 500 μg/mL into the 5 mL volumetric flask. Pipet 450 μL agluco vancomycin approximately 500 μg/mL into the 5 mL volumetric flask.

### Quantitation-Limit Check Solution (QL Check Solution)

Prepare the QL Check Solution containing approximately 0.45 μg/mL CDP-1Major (approximately 0.05% level relative to Vancomycin in impurities sample preparations). Pipet 128 μL des-amido-isoaspartate vancomycin 500 μg/mL into a 100 mL volumetric flask. Adjust to 100 mL with Diluent 1 and mix well. Protect from light by wrapping the flask in aluminum foil. Transfer an aliquot of approximately 100 μL of the solution to an HPLC vial for HPLC analysis. The remaining solution may be divided into approximately 500 μL aliquots and stored at −20° C. for future use.

### System Suitability Solution

Accurately weigh approximately 25.00±1.00 mg of SA-182-003-14-068 vancomycin hydrochloride inhalation bulk powder for system suitability. Quantitatively transfer to a 25 mL volumetric flask using Diluent 1. Adjust to 25 mL with Diluent 1. Mix thoroughly and label System Suitability Solution. Protect from light by wrapping the flask in aluminum foil. Transfer an aliquot of approximately 100 μL of the solution to an HPLC vial insert in an HPLC vial for HPLC analysis.

The remaining solution may be divided into approximately 100 μL portions and stored at −20° C. for future use.

### Sample Preparation

Impurities: Vancomycin Hydrochloride API (approximately 900 μg/mL)

Accurately weigh 22.50±1.00 mg of vancomycin hydrochloride API. Record the weight in mg to two decimal places. Quantitatively transfer to a 25 mL volumetric flask using Diluent 1. Adjust to 25 mL with Diluent 1. Mix thoroughly and label API 1A. Prepare two additional sample solutions and label API 2A and API 3A.

Pipet 5.0 mL of API 1A to a 50 mL volumetric flask, add approximately 15 mL Diluent 1, mix thoroughly, adjust to volume, mix thoroughly and label API 1B. Prepare two additional sample solutions from the API 2A and API 3A and label API 2B and API 3B, respectively.

Protect from light by wrapping the flasks in aluminum foil. Transfer aliquots of each sample preparation to separate HPLC vials. Store the flasks at 2-8° C., protect from light and assign an expiration date of 7 days from the date of preparation.

Impurities: Vancomycin hydrochloride inhalation powder (powder from bulk or from capsules, approximately 900 μg/mL)

For bulk powder, accurately weigh 25.00±1.00 mg of vancomycin hydrochloride inhalation powder from the bulk powder container. For powder from capsules, open 4 capsules and transfer the powder to a 20 mL scintillation vial. Weigh 25.00±1.00 mg of vancomycin hydrochloride inhalation powder from the scintillation vial. Record the weight in mg to two decimal places. Quantitatively transfer into a 25-mL volumetric flask using Diluent 1. Add approximately 15 mL Diluent 1, mix thoroughly until dissolved, adjust to volume with Diluent 1, mix thoroughly and label Powder 1A (or Capsule 1A). Prepare two additional sample solutions and label Powder 2A (or Capsule 2A) and Powder 3A (or Capsule 3A).

Pipet 5.0 mL from Powder 1A (or Capsule 1A) into a 50 mL volumetric flask. Dilute to 50 mL with Diluent 1, mix thoroughly, and label Powder 1B (or Capsule 1B). Prepare two additional solutions from Powder 2A (or Capsule 2A) and Powder 3A (or Capsule 2A) and label Powder 2B (or Capsule 2B) and Powder 3B (or Capsule 3B), respectively. Protect from light by wrapping the flasks in aluminum foil. Transfer aliquots of each sample solution to separate HPLC vials. Store the flasks at 2-8° C. and assign an expiration date of 7 days from the date of preparation.

### HPLC Conditions

### Instrument Set-Up

Before running samples, equilibrate the column and establish the correct instrument settings and the correct compositions of Mobile Phase A and Mobile Phase B as follows.

### Column Equilibration

Install the column onto the HPLC instrument and set the column oven to 40° C. Flush the column with 97% Mobile Phase B (Section 5.1.6) at 0.2 mL/min for at least 20 minutes but not more than 30 minutes, allowing the column to come to temperature during this time. Flush the column with 100% Mobile Phase A at 0.4 mL/min for at least 10 minutes but not more than 30 minutes.

### Instrument Settings: Gradient Profile

Before running samples establish the correct gradient profile as follows:

Set up the instrument with the initial setting shown in Table 5.

Inject the QL Check Solution and run the gradient with the gradient start times shown in Table 5 with a start time for the first gradient (ST₁) of 44 minutes.

Inject Standard Solution A and run the gradient with a start time for first gradient (ST₁) of 44 minutes and the other gradient start times as shown in Table 5.

Compare the chromatograms obtained with reference chromatograms.

Adjust the gradient start times (ST₁ to ST₆) in appropriate increments, keeping the difference between each start time the same as shown in Table 5 and re-inject the QL Check Solution and the Standard Solution A.

Compare the chromatograms obtained with the reference chromatograms and repeat until the separation between vancomycin and the gradient-related peak shown in the reference chromatogram is obtained.

Once a gradient start time for a particular column-instrument combination has been established, the conditions may be used as a starting point for future HPLC analyses using that particular column-instrument combination.

### Mobile Phase Composition

Before running samples establish the correct compositions of Mobile Phase A and Mobile Phase B as follows:

Starting with the compositions of Mobile Phase A and Mobile Phase B and the gradient profile established above, make one injection of the Impurities Marker Solution and one injection of the System Suitability Solution.

Calculate the resolution factor (R_{2,1}) for the following pairs of peaks in the System Suitability Solution, using the procedures outlined below as guide to the identifying the peaks in the System Suitability solution and using the following equation. If the electronic integrator is unable to measure the widths at half height of the two peaks, use the tangent-extrapolated width at half height for the larger of the two peaks for both w₁ and w₂.

a. CDP-1Major/vancomycin

b. Unknown Impurity 9/des-amido-succinimido-vancomycin

c. Des-amido-succinimido-vancomycin/unknown Impurity 10

d. Des-vancosamino-vancomycin/unknown impurity 22

$${R}_{1 , 2} = \frac{1.18 \times ( {t}_{2} - {t}_{1} )}{( {w}_{2} + {w}_{1} )}$$

where:
1.18=Constant
t₁=Retention time of the first peak in the critical pair of peaks
t₂=Retention time of the second peak in the critical pair of peaks
w₁=Width at half height of the first peak in the critical pair of peaks
w₂=Width at half height of the second peak in the critical pair of peaks

The mean value for the separation of CDP-1Major and vancomycin should be not less than 1.5. The mean value for the separation of unknown impurity 9 and des-amido-succinimido-vancomycin should be not less than 0.6. The mean value for the separation of des-amido-succinimido-vancomycin and impurity 10 should be not less than 0.6. The mean value for the separation of des-vancosamino-vancomycin and unknown impurity 22 should be not less than 0.8. If all the acceptance criteria are met, analysis can proceed.

If one or more of the acceptance criteria are not satisfied, adjust the pH of Mobile Phase A and/or Mobile Phase B.

It may be necessary to adjust the pH of Mobile Phase A by adjusting the concentration of acetic acid in Mobile Phase A in an iterative fashion until all the acceptance criteria are satisfied. For each iteration, a fresh Buffer A solution should be made and the THF Stock Solution and Mobile Phase A should be prepared from this fresh Buffer A.

If the R_{2,1} value of CDP-1Major and vancomycin is less than 1.5 increase the pH of Mobile Phase A by decreasing the concentration of acetic acid in Buffer A solution using Table 7 as a guide, up to a maximum of pH 4.15. If the value of unknown impurity 9 and des-amido-succinimido-vancomycin is less than 0.6 decrease the pH of Mobile Phase A by increasing the concentration of acetic acid in Buffer A, using Table 10 as a guide, down to a minimum of 4.05. If the value of R_{2,1} for the separation of des-amido-succimido-vancomycin and unknown 10 is less than 0.6 increase the pH of Mobile Phase A by decreasing the concentration of acetic acid in Buffer A, using Table 10 as a guide, up to a maximum of 4.15.

If value of R_{2,1} for the separation of des-vancosamino-vancomycin and unknown 12 is less than 0.8 increase the pH of Mobile Phase B by increasing the concentration of sodium acetate solution used in the preparation of mobile phase B up to a maximum of 150 mM.

TABLE 7
Target Weight of 1N Acetic Acid for Preparation of 2 Liters
of Buffer A used to Prepare pH-Adjusted Mobile Phase A

| pH | Target Weight 1N Acetic Acid (g) |
|---|---|
| 4.15 | 134.00 |
| 4.14 | 137.00 |
| 4.13 | 140.50 |
| 4.12 | 144.00 |
| 4.11 | 147.00 |
| 4.10 | 150.00 |
| 4.09 | 154.00 |
| 4.08 | 157.50 |
| 4.07 | 161.50 |
| 4.06 | 165.00 |
| 4.05 | 170.00 |

Repeat calibration steps, if necessary, until the acceptance criteria of the resolution factors are satisfied. If attempts to satisfy the acceptance criteria are unsuccessful, the column should be replaced. It may be necessary to adjust the compositions of both Mobile Phase A and Mobile Phase B in an iterative fashion until the resolution factors are achieved. If mobile phase composition is adjusted, the gradient start time should be re-evaluated as above to verify the gradient is still acceptable.

Once a mobile phase composition for a particular column-instrument combination has been established, the composition may be used as a starting point for future HPLC instrument setup evaluations using that particular column-instrument combination.

Sample Analysis

Analysis Sequence

Using the instrument settings and mobile phase compositions established above, inject the standards and samples according to the injection sequence in Table 8.

TABLE 8
Injection Sequence

| | Number of |
|---|---|
| | Injections |
| Solution | Per Vials |
| 1. Diluent 1 | 1 |
| 2. QL Check Solution | 1 |
| 3. Standard A | 6 |
| 4. Standard B | 1 |
| 5. Impurity Marker Solution | 2 |
| 6. System Suitability Solution | 2 |
| 7. Diluent 1 | 1 |
| 8. Sample* 1A | 1 |
| 9. Sample 2A | 1 |
| 10. Sample 3A | 1 |
| 11. Sample 1B | 1 |
| 12. Sample 2B | 1 |
| 13. Sample 3B | 1 |
| 14. Standard A (bracketing standard injection) | 1 |

*API, Powder or Capsule Samples depending of type of samples analyzed

System Suitability

Standard Precision

Calculate the mean and the percent relative standard deviation (% RSD) of the peak areas of the vancomycin peak in Standard A. The % RSD (n=6) of the vancomycin peak in Standard A initial injections of Standard A must be no more than 1.0% to accept the results.

### Check Standard Agreement

The check standard agreement between Standard A and Standard B must be 100.0%±2.0% to accept the results. Bracketing Standard Agreement: The bracketing standard agreement between the six initial Standard A injections and each Standard A bracketing standard injection after sample injections must 100.0%±2.0% to accept the results.

### Resolution (R_{2,1})

Calculate the mean (n=2) resolution factor (R_{2,1}) for the following pairs of peaks in the System Suitability Solution, using the equation above. The mean value for R_{2,1} for the separation of CDP-1Major and vancomycin should be not less than 1.5 to accept the results. The mean value for R_{2,1} for the separation of unknown impurity 9 and des-amido-succinimido-vancomycin should be not less than 0.6 to accept the results. The mean value for R_{2,1} for the separation of des-amido-succinimido-vancomycin and impurity 10 should be not less than 0.6 to accept the results. The mean value for R_{2,1} for the separation of des-vancosamino-vancomycin and unknown impurity 22 should be not less than 0.8 to accept the results. If all the System Suitability Criteria are satisfied, calculate the S/N ratio as described below. If one or more of the System Suitability Criteria are not satisfied, adjust the composition of Mobile Phase A or Mobile Phase B as described above.

### Signal-To-Noise Ratio

Calculate the signal-to-noise ratio (S/N) of CDP-1Major in the QL Check Solution (Table 11). The S/N should be not less than 5 to accept the results.

### Tailing Factor

Calculate the USP <621> Tailing Factor of the vancomycin peak in the first injection of Standard A. The USP Tailing Factor of the vancomycin peak Standard A should be between 0.85 and 1.15 to accept the results.

### Identification of Impurities

### Impurities in Impurities Marker Solution

The elution order of the six peaks in the Impurities Marker Solution is given in Table 9.

TABLE 9

| Elution Order | Classification |
|---|---|
| Des-amido-isoaspartate vancomycin (CDP-1minor) | Degradant |
| N-demethyl vancomycin | Drug Substance |
| | Impurity |
| Des-amido-isoaspartate vancomycin (CDP-1Major) | Degradant |
| Vancomycin | Drug Substance |
| Des-vancosamino vancomycin | Degradant |
| Agluco vancomycin | Degradant |

Identify vancomycin and the five impurities in the Impurities Marker Solution using the elution order in Table 9. Use the average RT of impurities in the Impurities Marker Solution Injections and the example chromatograms as a guide to identifying known impurities in Table 10 if present in sample chromatograms.

TABLE 10
Relative Response Factors (RRF) and Relative Retention Times
(RRT) of the Known and Unknown Impurities of Vancomycin

| | | | RRT | |
|---|---|---|---|---|
| | | | vs. | vs. |
| Impurity | Classification | RRF | NDMV | DVA |
| Unk 1 | Unidentified | 1.000 | 0.237 | NA |
| Unk 2 | Unidentified | 1.000 | 0.269 | NA |
| Unk 3 | Unidentified | 1.000 | 0.311 | NA |
| Unk 4 | Unidentified | 1.000 | 0.333 | NA |
| Unk 5 | Unidentified | 1.000 | 0.350 | NA |
| Des-amido-isoaspartyl- | Degradant | 1.000 | RS | RS |
| vancomycin (CDP-1Minor) | | | | |
| Unk 6 | Unidentified | 1.000 | 0.403 | NA |
| Unk 7 | Unidentified | 1.000 | 0.422 | NA |
| Unk 8 | Unidentified | 1.000 | 0.461 | NA |
| Unk 9 | Unidentified | 1.000 | 0.524 | NA |
| Des-amido-succinimido- | Degradant | 1.000 | 0.551 | NA |
| vancomycin | | | | |
| Unk 10 | Unidentified | 1.000 | 0.578 | NA |
| Unk 11 | Unidentified | 1.000 | 0.617 | NA |
| Unk 12 | Unidentified | 1.000 | 0.652 | NA |
| Unk 13 | Unidentified | 1.000 | 0.720 | NA |
| Unk 14 | Unidentified | 1.000 | 0.743 | NA |
| De-(L-Asn)-L-Gln- | API impurity | 1.000 | 0.772 | NA |
| vancomycin | | | | |
| Unk 15 | Unidentified | 1.000 | 0.806 | NA |
| Unk 16 | Unidentified | 1.000 | 0.858 | NA |
| Mono-dechloro-(2)- | API impurity | 1.000 | 0.948 | NA |
| vancomycin | | | | |
| N-demethyl-vancomycin | API impurity | 1.000 | RP1 | NA |
| Unk 17 | Unidentified | 1.000 | 1.056 | NA |
| Unk 18 | Unidentified | 1.000 | 1.105 | NA |
| Cis-chloro-(2)- | API impurity | 1.000 | 1.136 | NA |
| vancomycin | | | | |
| Des-amido-isoaspartyl- | Degradant | 1.000 | RS | RS |
| vancomycin (CDP-1Major) | | | | |
| Unk 19 | Unidentified | 1.000 | 1.308 | NA |
| Vancomycin | Active | NA | NA | NA |
| Unk 20 | Unidentified | 1.000 | NA | 0.901 |
| Unk 21 | Unidentified | 1.000 | NA | 0.921 |
| Mono-dechloro-(6)- | API impurity | 1.000 | NA | 0.944 |
| vancomycin | | | | |
| Des-vancosamino- | Degradant | 1.000 | NA | RP2 |
| vancomycin | | | | |
| Unk 22 | Unidentified | 1.000 | NA | 1.009 |
| N-Methyl-vancomycin | API impurity | 1.000 | NA | 1.017 |
| Unk 23 | Unknown | 1.000 | NA | 1.052 |
| Epi-26(R)-vancomycin | Degradant | 1.000 | NA | 1.111 |
| Agluco-vancomycin | Degradant | 1.000 | RS | RS |

RS: Reference Standard. The retention time of this peak in the samples is based on retention time of the reference standard in the Impurities Marker Solution
NA: Not applicable
RP1: Reference peak 1, used to calculate the retention times of the peaks eluting before vancomycin
RP2: Reference peak used to calculate the retention times of the peaks eluting after vancomycin

Retention times of impurities in the Impurities Marker Solution are used to predict retention times and identify the other known impurities not included in the Impurities Marker Solution as well as unknown impurities as described below.

### Predicted Retention Times (RT) of Other Known and Unknown Peaks

Except where indicated, compare the retention time of the peaks eluting before vancomycin with the expected retention times calculated using the relative retention times (RRTs) in Table 11 and the following equation:

RT(Predicted)=RT(NDMV)×RRT

Where:
RT (Predicted)=Predicted RT of the impurities eluting before vancomycin
RT (NDMV)=Average Retention Time of N-demethyl in the Impurities Marker Solution
RRT=Relative Retention Time of the impurity, relative to N-demethyl vancomycin (see Table 10)

Except where indicated, compare the retention time of the peaks eluting after vancomycin with the expected retention times calculated using the relative retention times (RRTs) in Table 11 and the following equation:

RT(Predicted)=RT(DVA)×RRT

Where:
RT (Predicted)=Predicted RT of the impurities eluting before vancomycin
RT (DVA)=Average Retention Time of desvancosamino vancomycin in the Impurities Marker Solution
RRT=Relative Retention Time of the impurity, relative to desvancosamino vancomycin (see Table 10)

Calculations

The calculated values are left un-rounded when used in subsequent calculations and calculations of averages, standard deviations, etc. to avoid the impact of multiple intermediate rounding steps on the final results.

Concentration of Standard A and Standard B Solution Preparation (Cws)

Calculate the concentration of vancomycin in Standard A and Standard B, as follows:

$${C}_{s} = \frac{W}{V} \times \frac{( 100 - M )}{100} \times P \times 1000 μg \text{/} mL$$

Where:
C=Concentrations of either Standard A or Standard B
W=Weight of either Standard A or Standard B
P=Purity of vancomycin (%), anhydrous, in Vancomycin HCl Standard
M=Moisture content of Vancomycin HCl Standard (measured at time of preparation)
V=Final volume of Standard A and Standard B Solution (250 mL)

Report the concentration of Vancomycin in Standard A and Standard B in μg/mL to 2 decimal places.

Impurity Concentration

Calculate the concentration of each individual impurities in the samples solutions 1A, 1B and 1C, as follows:

$${C}_{i} = \frac{( {A}_{i} ) \times {C}_{A}}{{A}_{A} \times {RRF}_{i}} μg \text{/} mL$$

Where:
Cᵢ=Concentration of impurity in sample solution 1A, 2A or 3A (in μg/mL)
Aᵢ=Area of each impurity in the injection of the sample solution
C_{A}=Concentration of vancomycin in Standard solution A (in μg/mL) (Section 10.1)
A_{A}=Average area of initial n=6 Standard A injections
RRFᵢ=Relative response factor of the impurity (see Table 10)

Calculate the total concentration of all the impurities in sample solutions 1A, 2A and 3A as follows (include only those impurities present at concentrations equal to or greater (≥) 0.45 μg/mL):

$${C}_{i , total} = {\sum }_{i = 1}^{i = n} {C}_{i} μg \text{/} mL$$

Where:
C_{i,total}=Total concentration of impurities in sample solutions 1A, 2A or 3A

Report the total impurity concentration (μg/mL) in impurity solutions 1A, 2A and 3A to three decimal places in the project notebook.

Concentration of Vancomycin in the Samples

Calculate the concentration of vancomycin the samples 1B, 2B and 3B as follows:

$${C}_{V} = \frac{{A}_{V} \times {C}_{A}}{{A}_{A}} μg \text{/} mL$$

Where:
C_{V}=Vancomycin concentration in sample solutions 1B, 2B or 3B (μg/mL)
A_{V}=Area of vancomycin in Sample 1B, 2B or 3B
C_{A}=Concentration of Standard A in μg/mL)
A_{A}=Average area of initial six Standard A injections

Concentration of Each Individual Impurity in the Sample (Impurity %)

Calculate the concentration of each individual impurity in samples 1A, 2A and 3A, as follows:

$$I {\%}_{i} = \frac{{C}_{i}}{( {C}_{V} \times 10 ) + {C}_{i , total}} \times 100 \%$$

Where:
I %ᵢ=Impurity concentration in sample 1A, 2A or 3A (as percentage)
10=Dilution factor (50 mL/5.0 mL)
C_{V}=Vancomycin concentration in sample solutions 1B, 2B or 3B (μg/mL)
Cᵢ=Concentration of impurity in sample solution 1A, 2A or 3A (μg/mL)
C_{i,total}=Total concentration of impurities in sample solutions 1A, 2A or 3A

Calculate the average concentration as a percentage of each individual impurity in the sample (vancomycin hydrochloride, API, vancomycin hydrochloride inhalation powder or capsules).

Total Concentration of Individual Impurities in the Sample

Calculate the total concentration of the impurities in the samples, as follows.

$$I {\%}_{total} = {\sum }_{i = 1}^{i = n} I {\%}_{i}$$

Where:
I %ₜₒₜₐₗ=Total impurity concentration in sample 1A, 2A or 3A (as percentage)
I %ᵢ=Impurity concentration in sample 1A, 2A or 3A (as percentage)

Include only the reportable impurities in the calculation of total reportable impurities.

Data Reporting

Impurities in Vancomycin Hydrochloride, API

Report the impurities having concentration that exceed the Reporting Threshold of 0.05%. Therefore, report all values equal to or greater than 0.06%. Report all values equal to or less that the reporting threshold as “≤0.05%”. For impurities below 1.0%, report value to two decimal places (e.g. 0.06%, 0.13%). At or above 1.0% report values to one decimal place (e.g. 1.0%, 1.3%).

Impurities Vancomycin Hydrochloride in Vancomycin Hydrochloride Inhalation Powder and Capsules

Report the impurities having concentration that exceed the Reporting Threshold of 0.05% in samples of vancomycin drug substance and greater than 0.1% in samples of AeroVanc.

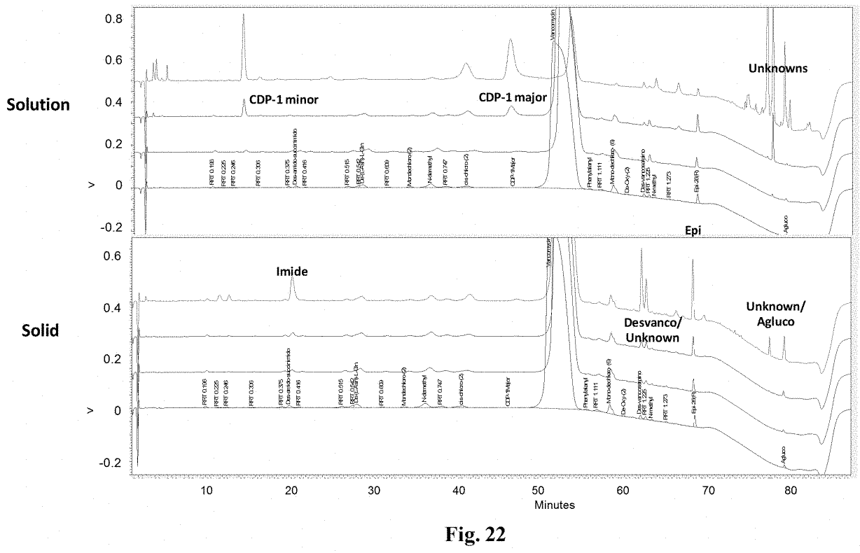

The method disclosed herein was successfully applied to the determination of degradation products of vancomycin in the solution and solid states. FIG. 22 shows the application of the method described herein to the separation of degraded samples of vancomycin in the solution and solid state to investigate impurity profiles of degraded vancomycin. Degradation of vancomycin in aqueous solution shows larger amounts of CDP-1 Minor, CPD-1 Major and additional unknowns eluting between 72 min and 84 min compared to the degraded solid. The degraded solid shows increased desamido-succinimide vancomycin and agluco vancomycin compared to the degraded aqueous solution of vancomycin.

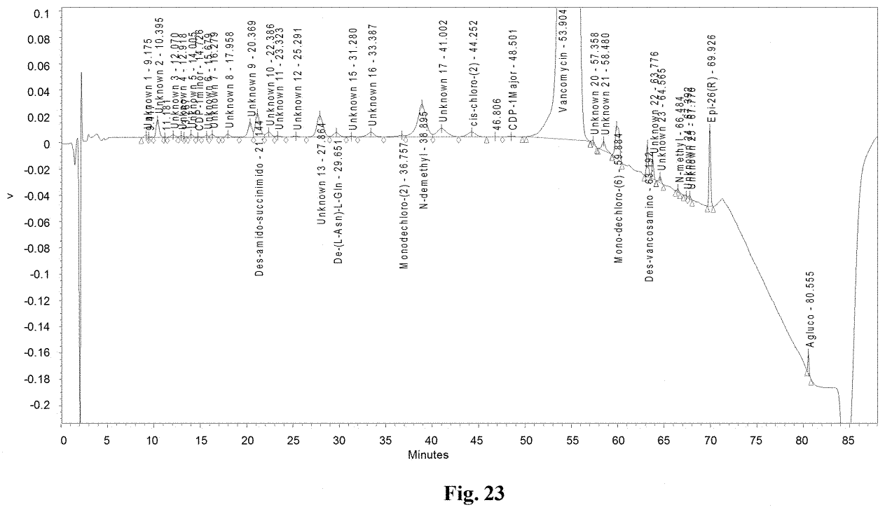

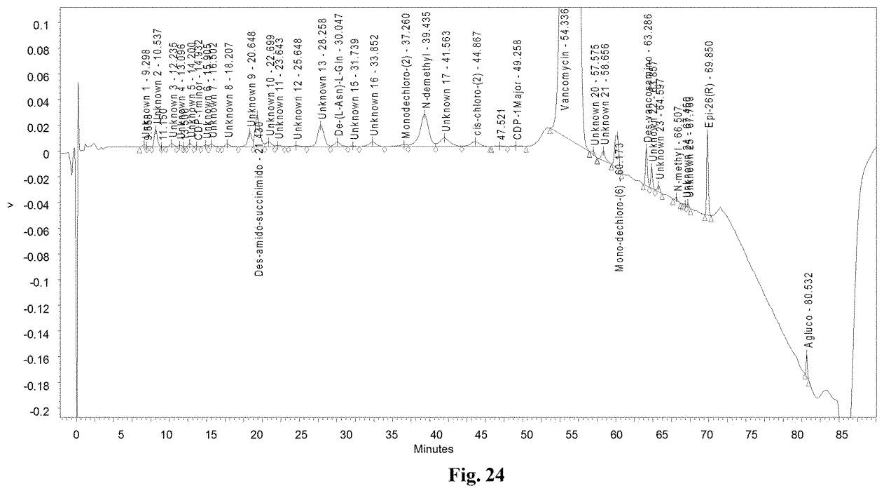

The method disclosed herein was also applied to the determination of degradation products of AeroVanc vancomycin powder stored for three months at 5° C. and 25° C. FIG. 23 shows a chromatogram of a sample of vancomycin powder stored at 5° C. and FIG. 24 shows a chromatogram of a sample of vancomycin powder stored at 25° C. Table 12 summarizes the degradation products and/or impurities in the samples. In general, the percentages of the impurities were either unchanged or slightly higher in the sample stored at 25° C.

TABLE 12

| | 5° C. | | 25° C. | |
|---|---|---|---|---|
| | Retention | | Retention | |
| | Time | Impurity | Time | Impurity |
| Name | (min) | (%) | (min) | (%) |
| Unknown 1 | 9.175 | 0.040 | 9.298 | 0.040 |
| RRT = 0.175 | 9.417 | 0.011 | 9.558 | 0.012 |
| Unknown 2 | 10.395 | 0.294 | 10.537 | 0.293 |
| RRT = 0.207 | 11.181 | 0.003 | 11.150 | 0.005 |
| Unknown 3 | 12.070 | 0.058 | 12.235 | 0.076 |
| Unknown 4 | 12.918 | 0.033 | 13.096 | 0.034 |
| RRT = 0.246 | 13.267 | 0.010 | 13.500 | 0.008 |
| Unknown 5 | 14.005 | 0.052 | 14.200 | 0.061 |
| CDP-1minor | 14.726 | 0.017 | 14.932 | 0.026 |
| Unknown 6 | 15.679 | 0.039 | 15.905 | 0.043 |
| Unknown 7 | 16.279 | 0.062 | 16.502 | 0.063 |
| Unknown 8 | 17.958 | 0.075 | 18.207 | 0.077 |
| Unknown 9 | 20.369 | 0.328 | 20.648 | 0.322 |
| Des-amido-succinimido | 21.144 | 0.577 | 21.430 | 0.759 |
| Unknown 10 | 22.386 | 0.151 | 22.699 | 0.149 |
| Unknown 11 | 23.323 | 0.040 | 23.643 | 0.042 |
| Unknown 12 | 25.291 | 0.039 | 25.648 | 0.038 |
| Unknown 13 | 27.864 | 0.755 | 28.258 | 0.744 |
| De-(L-Asn)-L-Gln | 29.651 | 0.152 | 30.047 | 0.151 |
| Unknown 15 | 31.280 | 0.020 | 31.739 | 0.020 |
| Unknown 16 | 33.387 | 0.186 | 33.852 | 0.185 |
| Monodechloro-(2) | 36.757 | 0.069 | 37.260 | 0.072 |
| N-demethyl | 38.895 | 1.757 | 39.435 | 1.722 |
| Unknown 17 | 41.002 | 0.468 | 41.563 | 0.460 |
| Unknown 18 | 42.521 | 0.000 | 42.521 | 0.000 |
| cis-chloro-(2) | 44.252 | 0.194 | 44.867 | 0.200 |
| RRT = 0.868 | 46.806 | 0.008 | 47.521 | 0.008 |
| CDP-1Major | 48.501 | 0.022 | 49.258 | 0.021 |
| Unknown 19 | 50.332 | 0.000 | 50.332 | 0.000 |
| Vancomycin | 53.904 | — | 54.336 | — |
| Unknown 20 | 57.358 | 0.050 | 57.575 | 0.056 |
| Unknown 21 | 58.480 | 0.197 | 58.656 | 0.205 |
| Mono-dechloro-(6) | 59.884 | 0.679 | 60.173 | 0.671 |
| Des-vancosamino | 63.192 | 0.343 | 63.286 | 0.398 |
| Unknown 22 | 63.776 | 0.188 | 63.857 | 0.205 |
| Unknown 23 | 64.565 | 0.072 | 64.597 | 0.083 |
| N-methyl | 66.484 | 0.019 | 66.507 | 0.027 |
| Unknown 24 | 67.392 | 0.018 | 67.450 | 0.016 |
| Unknown 25 | 67.770 | 0.034 | 67.763 | 0.035 |
| Epi-26(R) | 69.926 | 0.610 | 69.850 | 0.628 |
| Unknown 26 | 78.756 | 0.000 | 78.756 | 0.000 |
| Agluco | 80.555 | 0.139 | 80.532 | 0.163 |
| Total | | 7.808 | | 8.119 |

We claim:

1. A method for separating a mixture of compounds each having at least one ionizable group, the method comprising: treating the mixture with reversed phase high pressure liquid chromatography on a stationary phase comprising a reversed phase chromatography column in the presence of a mobile phase comprising (a) a first mobile phase component comprising an aqueous buffer system comprising a weak acid and optionally a conjugate base, and at least one organic solvent miscible with water, and (b) a second mobile phase component comprising an aqueous buffer system comprising a conjugate base and optionally a weak acid, and a different concentration of at least one organic solvent miscible with water, wherein the ratio of weak acid to conjugate base in the first mobile phase component is different from the ratio of weak acid to conjugate base in the second mobile phase component and the ratio of the first mobile phase component to the second mobile phase component is varied during the separation.

2. The method of claim 1 wherein the at least one miscible organic solvent is selected from the group consisting of alcohols, acetonitrile, dioxane, tetrahydrofuran and mixtures thereof.

3. The method of claim 2 wherein the at least one miscible organic solvent is selected from the group consisting of tetrahydrofuran, acetonitrile and mixtures thereof.

4. The method of claim 3 wherein the first mobile phase component comprises tetrahydrofuran and acetonitrile and the second mobile phase component comprises tetrahydrofuran and acetonitrile in different percentages than in the first mobile phase component.

5. The method of claim 1 wherein the buffer systems comprise formic acid, sodium formate, sodium acetate, acetic acid, ammonium formate, ammonium acetate or combinations thereof.

6. The method of claim 5 wherein the buffer systems comprise sodium acetate and acetic acid.

7. The method of claim 6 wherein the first mobile phase component comprises acetic acid and sodium acetate as buffer components and wherein the second mobile phase component comprises sodium acetate and optionally acetic acid as buffer components in a different ratio than in the first mobile phase component.

8. The method of claim 7 wherein the second mobile phase component comprises sodium acetate and no acetic acid as buffer components.

9. The method of claim 1 wherein the pH of the first mobile phase component is controlled between 2 and 8.5.

10. The method of claim 1 wherein the pH of the second mobile phase component is controlled between 2.5 and 9.5.

11. The method of claim 1 wherein the reversed phase chromatography column comprises a stationary phase comprising silica gel particles having a chemically modified surface comprising octadecyl-pentafluorophenyl-silyl (C18 PFP) moieties.

12. The method of claim 1 wherein the second mobile phase component comprises 0% of the total mobile phase for an initial period of time and is then increased in one or more linear ramps to comprise a major portion of the total mobile phase over a second period of time.

13. The method of claim 1 wherein the second mobile phase component comprises 0% of the total mobile phase for an initial period of time and is then increased in a single step to comprise a major portion of the total mobile phase over a second period of time.

14. The method of claim 1 comprising quantifying the amount of each of the compounds in the mixture.

15. The method of claim 1 comprising identifying at least one impurity in the mixture.

16. The method of claim 1 wherein each of the compounds in the mixture has at least two ionizable groups.

17. The method of claim 1 wherein the mixture of compounds comprises a glycopeptide antibiotic and one or more impurities or degradation products.

18. The method of claim 17 wherein the mixture of compounds comprises vancomycin or vancomycin hydrochloride, and one or more impurities.

19. The method of claim 18 wherein the mixture further comprises leucine.

20. The method of claim 18 wherein at least one impurity in a vancomycin composition can be measured in the range of 0.05% to 10.0% relative to vancomycin.
